(19) United States
(12) Patent Application Publication
(10) Pub. No.: US 2013/0030170 A1
(43) Pub. Date: Jan. 31, 2013

(54) ENANTIOSELECTIVE CYCLOPROPENATION OF ALKYNES

(71) Applicants: X. Peter Zhang, Tampa, FL (US); Xin Cui, Tampa, FL (US)

(72) Inventors: X. Peter Zhang, Tampa, FL (US); Xin Cui, Tampa, FL (US)

(73) Assignee: UNIVERSITY OF SOUTH FLORIDA, Tampa, FL (US)

(21) Appl. No.: 13/364,570

(22) Filed: Feb. 2, 2012

Related U.S. Application Data
(60) Provisional application No. 61/439,674, filed on Feb. 4, 2011.

(51) Int. Cl. C07C 253/30; C07D 487/22; C07C 1/20; C07C 1/32; C07C 5/44; C07C 1/26

(52) U.S. Cl. 540/145; 585/365; 585/357; 558/366

(57) ABSTRACT

The cobalt(II) complex of new D2-symmetric chiral porphyrin 3,5-DiMes-ChenPhyrin, [Co(P2)], has been shown to be a highly effective chiral metalloradical catalyst for enantioselective cyclopropenation of alkynes with acceptor/acceptor-substituted diazo reagents such as α-cyanodiazoacetamides and α-cyanodiazoacetates. The [Co(P2)]-mediated metalloradical cyclopropenation is suitable to a wide range of terminal aromatic and related conjugated alkynes with varied steric and electronic properties, providing the corresponding tri-substituted cyclopropenes in high yields with excellent enantiocontrol of the all-carbon quaternary stereogenic centers. In addition to mild reaction conditions, the Co(II)-based metalloradical catalysis for cyclopropenation features a high degree of functional group tolerance.

### CROSS-REFERENCE TO RELATED APPLICATIONS

The present application claims the benefit of U.S. Provisional Application Ser. No. 61/439,674, filed Feb. 4, 2011, which is hereby incorporated by reference herein in its entirety.

### STATEMENT OF GOVERNMENT RIGHTS

This invention was made with Government support under grant number NSF #0711024, awarded by the National Science Foundation, Division of Chemistry. The Government has certain rights in the invention.

### FIELD OF THE INVENTION

The present invention generally relates to Co(II)-based metalloradical-catalyzed cyclopropenation of alkynes. More particularly, the present invention relates to a process for asymmetric cyclopropenation of alkynes with a Co(II) porphyrin complex catalyst and an acceptor/acceptor-substituted diazo reagent.

### BACKGROUND OF THE INVENTION

Cyclopropenes are a unique class of carbocyclic compounds with unsaturated, highly strained three-membered ring structures. The combination of high strain and unsaturation renders cyclopropenes as versatile synthons for a wide variety of synthetic organic transformation.¹ Consequently, significant efforts have been devoted toward the synthesis of this class of molecules, especially optically active chiral cyclopropenes.^{1,2} Of different methods, catalytic asymmetric cyclopropenation of alkynes with diazo reagents constitutes one of the most direct and general methods for stereoselective construction of this type of strained ring structures.^{1,2} A number of catalytic systems based on dirhodium(II) complexes of chiral carboxamidate and carboxylate ligands have been successfully developed to catalyze enantioselective cyclopropenation using several different types of diazo reagents as carbene sources, including diazoacetates,^{3,4} diazosulfones,⁵ aryldiazoacetates,⁶ and styryldiazoacetates.^{7,8,9} While the existing chiral Rh₂ catalysts were shown to be highly effective with both acceptor- and donor/acceptor-substituted diazo regents, asymmetric cyclopropenation with acceptor/acceptor-substituted diazo reagents remains to be developed.¹⁰ Owing to the presence of two electron-withdrawing groups at the α-carbon, this class of diazo reagents has inherent low reactivity with Lewis acidic metal catalysts toward formation of the corresponding metallocarbene intermediates. Even when they can be formed under forcing conditions, their subsequent reactions with substrates are often difficult in terms of controlling enantioselectivity due to the high electrophilicity of the acceptor/acceptor-substituted metallocarbenes.

As stable metalloradicals with well-defined open-shell doublet d⁷ electronic structure, cobalt(II) complexes of porphyrins, [Co(Por)], have emerged as a new class of carbene transfer catalysts for olefin cyclopropanation.¹¹ With the introduction of D₂-symmetric chiral porphyrins as supporting ligands,^{11a,12} the Co(II)-based metalloradical catalysts [Co(D₂-Por*)] have been demonstrated to be highly effective for asymmetric cyclopropanation of a broad combination of olefin substrates and diazo reagents with excellent diastereo- and enantioselectivity,¹³ including electron-deficient olefins^{13b} and acceptor/acceptor-substituted diazo reagent.^{13d,13g} It is evident that Co(II)-based metalloradical cyclopropanation possesses a distinct reactivity profile from the widely studied Rh₂- and Cu-based closed-shell systems. Recent detailed mechanistic study confirmed the involvement of an unusual Co(III)-carbene radical as the key intermediate and elucidated an unprecedented stepwise radical addition-substitution pathway for the Co(II)-catalyzed olefin cyclopropanation.¹⁴

### SUMMARY OF THE INVENTION

To further validate the concept of metalloradical catalysis (MRC), we envisioned the possibility of Co(II)-based catalytic process for alkyne cyclopropenation if the radical addition-substitution pathway of the Co(III)-carbene radical intermediate could be also operative for alkynes in a similar way to alkenes. To address the aforesaid challenges in the area, we decided to target α-cyanodiazoacetates and α-cyanodiazoacetamides, two types of common acceptor/acceptor-substituted diazo reagents that have not been previously applied for asymmetric cyclopropenation (Scheme 1).¹⁰ These catalytic processes would be synthetically attractive as the resultant multi-functionalized cyclopropenes bearing an all-carbon quaternary stereogenic center can serve as invaluable chiral synthons for a range of stereoselective synthetic applications as illustrated by Scheme 1.^{1,2}

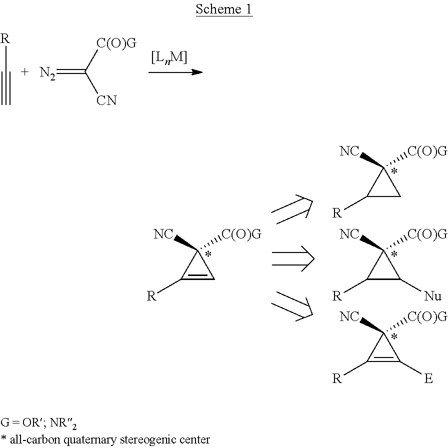

As the outcome of the effort, we report herein a highly efficient catalytic system based on a new chiral Co(II) metalloradical catalyst for enantioselective cyclopropenation of alkynes with both α-cyanodiazoacetates and α-cyanodiazoacetamides. In addition to high enantioselectivity, the Co(II)-catalyzed cyclopropenation can operate at room temperature using a stoichiometric ratio of reactants without the need of slow addition of the diazo reagents. Furthermore, the metalloradical catalytic process features a remarkable degree of tolerance toward various functionalities, including CHO, OH and NH₂ groups.

One aspect of the present invention is a process for the preparation for the preparation of a cyclopropene. The process comprises treating an alkyne with an acceptor/acceptor substituted diazo reagent in the presence of a metal porphyrin complex.

Another aspect of the present invention is a cyclopentene corresponding to Formula C-1

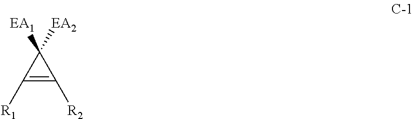

wherein

R₁ is hydrocarbyl, substituted hydrocarbyl, heterocyclo, or an electron withdrawing group,

R₂ is hydrogen, substituted hydrocarbyl, or heterocyclo, and

EA₁ and EA₂ are the same or different, and each is an electron-acceptor.

Another aspect of the present invention is a cobalt porphyrin complex corresponding to Formula [Co(P2)]

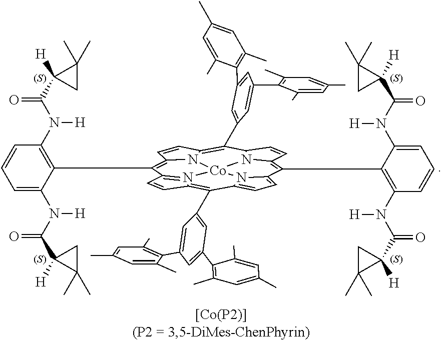

Other objects and features will be in part apparent and in part pointed out hereinafter.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

The term “acyl,” as used herein alone or as part of another group, denotes the moiety formed by removal of the hydroxyl group from the group —COOH of an organic carboxylic acid, e.g., RC(O)—, wherein R is R¹, R¹O—, R¹R²N— or R¹S—, R¹ is hydrocarbyl, heterosubstituted hydrocarbyl, or heterocyclo and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The terms “alkoxy” or “alkoxyl” as used herein alone or as part of another group denote any univalent radical, RO— where R is an alkyl group.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like. The substituted alkyl groups described herein may have, as substituents, any of the substituents identified as substituted hydrocarbyl substituents.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, as phenyl, biphenyl, naphthyl, substituted phenyl, substituted biphenyl or substituted naphthyl. Phenyl and substituted phenyl are the more preferred aryl. The substituted aryl groups described herein may have, as substituents, any of the substituents identified as substituted hydrocarbyl substituents.

The terms “diazo” or “azo” as used herein alone or as part of another group denote an organic compound with two linked nitrogen compounds. These moieties include without limitation diazomethane, ethyl diazoacetate, and t-butyl diazoacetate.

The term “electron acceptor” as used herein denotes a chemical moiety that accepts electrons. Stated differently, an electron acceptor is a chemical moiety that accepts either a fractional electronic charge from an electron donor moiety to form a charge transfer complex, accepts one electron from an electron donor moiety in a reduction-oxidation reaction, or accepts a paired set of electrons from an electron donor moiety to form a covalent bond with the electron donor moiety.

The terms “halogen” or “halo” as used herein alone or as part of another group denote chlorine, bromine, fluorine, and iodine.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The term “heteroatom” as used herein denotes atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainded of the molecule through a carbon or heteroatom. Exemplary heterocyclo include heteroaromatics as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein alone or as part of another group denote organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, as alkaryl, alkenaryl, and alkynaryl. Unless otherwise indicated, these moieties preferably comprise 1 to 20 carbon atoms.

The term “porphyrin” refers to a compound comprising a fundamental skeleton of four pyrrole nuclei united through the α-positions by four methane groups to form the following macrocyclic structure:

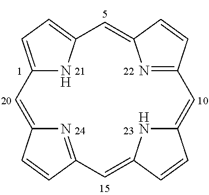 [SMILES: C1=Cc2cc3ccc(cc4nc(cc5ccc(cc1n2)[nH]5)C=C4)[nH]3]

The “substituted hydrocarbyl” moieties described herein, e.g., the substituted alkyl, the substituted alkenyl, the substituted alkynyl, and the substituted aryl moieties, are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substitutents include halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers.

### DETAILED DESCRIPTION OF THE PREFERRED EMBODIMENTS

In accordance with the process of the present invention, compounds containing an acetylenic bond, commonly known as alkynes, are cyclopropenated with a diazo reagent in the presence of a metal porphyrin complex. As described and exemplified in greater detail herein, certain metal porphyrin complexes are a highly effective chiral metalloradical catalyst for enantioselective cyclopropenation of alkynes with acceptor/acceptor-substituted diazo reagents. In one embodiment, the metal porphyrin complex is a cobalt(II) complex of a D₂-symmetric chiral porphyrin such as 3,5-DiMes-ChenPhyrin, (Co(P2)) and the acceptor/acceptor-substituted diazo reagent is a diazo reagent such as an α-cyanodiazoacetamides or an α-cyanodiazoacetates. The cobalt(II) complex-mediated metalloradical cyclopropenation is suitable to a wide range of terminal aromatic and related conjugated alkynes with varied steric and electronic properties, providing the corresponding tri-substituted cyclopropenes in high yields with excellent enantiocontrol of the all-carbon quaternary stereogenic centers. In addition to mild reaction conditions, the Co(II)-based metalloradical catalysis for cyclopropenation features a high degree of functional group tolerance.

Alkyne Substrates

In general, a wide range of alkynes may be used as substrates to form cyclopropenes of the present invention. In one embodiment, the alkyne corresponds to

Formula A-1:

R₁—≡—R₂ Formula A-1

wherein R₁ is hydrocarbyl, substituted hydrocarbyl, heterocyclo, or an electron withdrawing group, and R₂ is hydrogen, substituted hydrocarbyl, or heterocyclo. For example, in one embodiment, R₁ may be alkyl or substituted alkyl. By way of further example, R₁ may be aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl. For example, R₁ may be —C(O)R, —C(O)OR, or —C(O)NRₐR_{b} wherein R, Rₐ and R_{b} are independently optionally substituted alkyl or optionally substituted aryl. By way of further example, R₁ may be heterocyclo; for example, R₁ may be optionally substituted furyl, thienyl, pyridyl or other five or six membered heterocyclo. In one embodiment, R₂ is hydrogen. In another embodiment, R₂ is alkyl or substituted alkyl. In yet another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro.

When the alkyne corresponds to Formula A-1 and R₁ is an electron withdrawing group, the olefin corresponds to Formula A-1-EWG:

EWG—≡—R₂ Formula A-1-EWG

wherein R₁ is an electron withdrawing group and R₂ is as defined in connection with Formula A-1. That is, R₂ is hydrogen, hydrocarbyl, substituted hydrocarbyl, or heterocyclo. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro.

In general, the alkyne's electron withdrawing group, EWG, as depicted in Formula A-1-EWG and described in connection with Formula A-1, is any substituent that draws electrons away from the ethylenic bond. Exemplary electron withdrawing groups include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron withdrawing group(s) is/are hydroxy, alkoxy, mercapto, halogen, carbonyl, sulfonyl, nitrile, quaternary amine, nitro, or trihalomethyl. In another embodiment, the electron withdrawing group(s) is/are halogen, carbonyl, nitrile, quaternary amine, nitro, or trihalomethyl. In another embodiment, the electron withdrawing group(s) is/are halogen, carbonyl, nitrile, nitro, or trihalomethyl. When the electron withdrawing group is alkoxy, it generally corresponds to the formula —OR where R is hydrocarbyl, substituted hydrocarbyl, or heterocyclo. When the electron withdrawing group is mercapto, it generally corresponds to the formula —SR where R is hydrogen, hydrocarbyl, substituted hydrocarbyl or heterocyclo. When the electron withdrawing group is a halogen atom, the electron withdrawing group may be fluoro, chloro, bromo, or iodo; typically, it will be fluoro or chloro. When the electron withdrawing group is a carbonyl, it may be an aldehyde (—C(O)H), ketone (—C(O)R), ester (—C(O)OR), acid (—C(O)OH), acid halide (—C(O)X), amide (—C(O)NEₐE_{b}), or anhydride (—C(O)OC(O)R) where R is hydrocarbyl, substituted hydrocarbyl or heterocyclo, Eₐ and E_{b} are independently hydrogen, hydrocarbyl, substituted hydrocarbyl or heterocyclo, and X is a halogen atom. When the electron withdrawing group is a sulfonyl, it may be an acid (—SO₃H) or a derivative thereof (—SO₂R) where R is hydrocarbyl, substituted hydrocarbyl or heterocyclo. When the electron withdrawing group is a quaternary amine, it generally corresponds to the formula —N⁺EₐE_{b}E_{c} where Eₐ, E_{b} and E_{c} are independently hydrogen, hydrocarbyl, substituted hydrocarbyl or heterocyclo. When the electron withdrawing group is a trihalomethyl, it is preferably trifluoromethyl or trichloromethyl. In each of the foregoing exemplary electron withdrawing groups containing the variable “X”, in one embodiment, X may be chloro or fluoro, preferably fluoro. In each of the foregoing exemplary electron withdrawing groups containing the variable “R”, R may be alkyl. In each of the foregoing exemplary electron withdrawing groups containing the variable “Eₐ” and “E_{b}”, and “E_{c},” Eₐ, E_{L}, and E_{c} may independently be hydrogen or alkyl.

In accordance with one preferred embodiment, the electron withdrawing group, if present, is a halide, aldehyde, ketone, ester, carboxylic acid, amide, acyl chloride, trifluoromethyl, nitrile, sulfonic acid, ammonia, amine, or a nitro group. In this embodiment, the electron withdrawing group(s) correspond to one of the following chemical structures: —X, —C(O)H, —C(O)R, —C(O)OR, —C(O)OH, —C(O)X, —C(X)₃, —CN, —SO₃H, —N⁺H₃, —N⁺R₃, or —N⁺O₂⁺where R is hydrocarbyl, substituted hydrocarbyl or heterocyclo and X is halogen.

In one preferred embodiment, the alkyne corresponds to Formula A-2:

R₁—≡— Formula A-2

wherein R₁ is hydrocarbyl, substituted hydrocarbyl, heterocyclo, or an electron withdrawing group. For example, in one embodiment, R₁ may be alkyl or substituted alkyl. By way of further example, R₁ may be aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl. For example, R₁ may be —C(O)R, —C(O)OR, or —C(O)NRₐR_{b} wherein R, Rₐ and R_{b} are independently optionally substituted alkyl or optionally substituted aryl. By way of further example, R₁ may be heterocyclo; for example, R₁ may be optionally substituted furyl, thienyl, pyridyl or other five or six membered heterocyclo.

Diazo Reagents

In one embodiment, the diazo reagent is an acceptor/acceptor-substituted diazo reagent corresponding to Formula D-1:

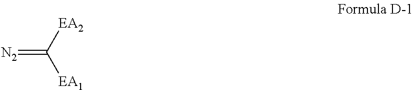

wherein EA₁ and EA₂ are the same or different, and each is an electron-acceptor. Exemplary electron-acceptors include hydroxy, alkoxy, mercapto, halogens, carbonyls, sulfonyls, nitrile, quaternary amines, nitro, trihalomethyl, imine, amidine, oxime, thioketone, thioester, or thioamide. For example, in one embodiment, EA₁ and EA₂ are independently hydroxy, alkoxy, mercapto, halogen, carbonyl, sulfonyl, nitrile, quaternary amine, nitro, or trihalomethyl. In another embodiment, EA₁ and EA₂ are independently halogen, carbonyl, nitrile, quaternary amine, nitro, or trihalomethyl. In another embodiment, EA₁ and EA₂ are independently halogen, carbonyl, nitrile, nitro, or trihalomethyl. When EA₁ and/or EA₂ is/are alkoxy, it/they generally correspond(s) to the formula —OR where R is hydrocarbyl, substituted hydrocarbyl, or heterocyclo. When EA₁ and/or EA₂ is/are mercapto, it/they generally correspond(s) to the formula —SR where R is hydrogen, hydrocarbyl, substituted hydrocarbyl or heterocyclo. When EA₁ and/or EA₂ is/are halogen, it/they may be fluoro, chloro, bromo, or iodo; typically, it/they will be fluoro or chloro. When EA₁ and/or EA₂ is/are carbonyl, it/they may independently be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl, alkoxy or heterocyclo, and X is a halogen atom. When the EA₁ and/or EA₂ is/are sulfonyl, it/they may independently be an acid (—SO₃H) or a derivative thereof (—SO₂R) where R is hydrocarbyl, substituted hydrocarbyl or heterocyclo. When EA₁ and/or EA₂ is/are quaternary amine, it/they generally correspond(s) to the formula —N⁺RₐR_{b}R_{c} where Rₐ, R_{b} and R_{c} are independently hydrogen, hydrocarbyl, substituted hydrocarbyl, alkoxy or heterocyclo. When EA₁ and/or EA₂ is/are trihalomethyl, it/they is/are preferably trifluoromethyl or trichloromethyl. In each of the foregoing exemplary electron-acceptors containing the variable “X”, in one embodiment, X may be chloro or fluoro, preferably fluoro. In each of the foregoing exemplary electron-acceptors containing the variable “R”, R may be alkyl. In each of the foregoing exemplary electron-acceptors containing the variable “Rₐ” and “R_{b}”, Rₐ and R_{b} may independently be hydrogen or alkyl.

In one embodiment, the diazo reagent is an acceptor/acceptor-substituted diazo reagent corresponding to Formula D-1 and EA₁ and EA₂ are the same. For example, EA₁ and EA₂ may each be the same and an electron-acceptor selected from among hydroxy, alkoxy, mercapto, halogen, carbonyl, sulfonyl, nitrile, quaternary amine, nitro, trihalomethyl, imine, amidine, oxime, thioketone, thioester, or thioamide. For example, in one embodiment, EA₁ and EA₂ are the same and are hydroxy, alkoxy, mercapto, halogen, carbonyl, sulfonyl, nitrile, quaternary amine, nitro, or trihalomethyl. In another embodiment, EA₁ and EA₂ are the same and are halogen, carbonyl, nitrile, quaternary amine, nitro, or trihalomethyl. In another embodiment, EA₁ and EA₂ are the same and are halogen, carbonyl, nitrile, nitro, or trihalomethyl. When in one embodiment, EA₁ and EA₂ are alkoxy, they generally correspond to the formula —OR where R is hydrocarbyl, substituted hydrocarbyl, or heterocyclo. When EA₁ and EA₂ are mercapto, they generally correspond to the formula —SR where R is hydrogen, hydrocarbyl, substituted hydrocarbyl or heterocyclo. When EA₁ and EA₂ are a halogen atom, they may be fluoro, chloro, bromo, or iodo; typically, they will be fluoro or chloro. When EA₁ and EA₂ are the same and are a carbonyl, they may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl, alkoxy or heterocyclo, and X is a halogen atom. When EA₁ and EA₂ are the same and are a sulfonyl, they may be an acid (—SO₃H) or a derivative thereof (—SO₂R) where R is hydrocarbyl, substituted hydrocarbyl or heterocyclo. When EA₁ and EA₂ are the same and are a quaternary amine, they generally correspond to the formula —N⁺RₐR_{b}R_{c} where Rₐ, R_{b} and R_{c} are independently hydrogen, hydrocarbyl, substituted hydrocarbyl, alkoxy or heterocyclo. When the EA₁ and EA₂ are the same and are a trihalomethyl, they are preferably trifluoromethyl or trichloromethyl. In each of the foregoing exemplary electron-acceptors containing the variable “X”, in one embodiment, X may be chloro or fluoro, preferably fluoro. In each of the foregoing exemplary electron-acceptors containing the variable “R”, R may be alkyl. In each of the foregoing exemplary electron-acceptors containing the variable “Rₐ” and “R_{b}”, Rₐ and R_{b} may independently be hydrogen or alkyl.

In one embodiment, the diazo reagent is an acceptor/acceptor-substituted diazo reagent corresponding to Formula D-1 and EA₁ and EA₂ are different. For example, EA₁ and EA₂ may be a different electron-acceptor selected from among hydroxy, alkoxy, mercapto, halogen, carbonyl, sulfonyl, nitrile, quaternary amine, nitro, trihalomethyl, imine, amidine, oxime, thioketone, thioester, and thioamide. For example, in one embodiment, EA₁ and EA₂ are different and selected from hydroxy, alkoxy, mercapto, halogen, carbonyl, sulfonyl, nitrile, quaternary amine, nitro, and trihalomethyl. In another embodiment, EA₁ and EA₂ are different and selected from halogen, carbonyl, nitrile, quaternary amine, nitro, and trihalomethyl. In another embodiment, EA₁ and EA₂ are different and selected from halogen, carbonyl, nitrile, nitro, and trihalomethyl. When EA₁ or EA₂ is alkoxy, it generally corresponds to the formula —OR where R is hydrocarbyl, substituted hydrocarbyl, or heterocyclo. When EA₁ or EA₂ is mercapto, it generally corresponds to the formula —SR where R is hydrogen, hydrocarbyl, substituted hydrocarbyl or heterocyclo. When EA₁ or EA₂ is a halogen atom, it may be fluoro, chloro, bromo, or iodo; typically, it will be fluoro or chloro. When EA₁ or EA₂ is a carbonyl, it may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl, alkoxy or heterocyclo, and X is a halogen atom. When EA₁ or EA₂ is a sulfonyl, it may be an acid (—SO₃H) or a derivative thereof (—SO₂R) where R is hydrocarbyl, substituted hydrocarbyl or heterocyclo. When EA₁ or EA₂ is a quaternary amine, it generally corresponds to the formula —N⁺RₐR_{b}R_{c} where Rₐ, R_{b} and R_{c} are independently hydrogen, hydrocarbyl, substituted hydrocarbyl, alkoxy or heterocyclo. When the EA₁ or EA₂ is a trihalomethyl, it preferably os trifluoromethyl or trichloromethyl. In each of the foregoing exemplary electron-acceptors containing the variable “X”, in one embodiment, X may be chloro or fluoro, preferably fluoro. In each of the foregoing exemplary electron-acceptors containing the variable “R”, R may be alkyl. In each of the foregoing exemplary electron-acceptors containing the variable “Rₐ” and “R_{b}”, Rₐ and R_{b} may independently be hydrogen or alkyl.

In accordance with one preferred embodiment, the diazo reagent corresponds to Formula D-1, and EA₁ and EA₂ are independently a halide, aldehyde, ketone, ester, carboxylic acid, amide, acyl chloride, trifluoromethyl, nitrile, sulfonic acid, ammonia, or an amine. For example, in this embodiment, EA₁ and EA₂ may be independently selected from the group consisting of —X, —C(O)H, —C(O)R, —C(O)OR, —C(O)OH, —C(O)X, —C(O)NRₐR_{b}, —C(X)₃, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is a halogen atom. By way of further example, in one such embodiment EA₁ and EA₂ are independently selected from the group consisting of —C(O)H, —C(O)R, —C(O)OR, —C(O)OH, —C(O)X, —C(O)NRₐR_{b}, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen.

In accordance with one preferred embodiment, the diazo reagent corresponds to formula D-2

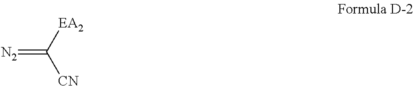

where EA₂ is hydroxy, alkoxy, mercapto, halogen, carbonyl, sulfonyl, nitrile, quaternary amine, nitro, trihalomethyl, imine, amidine, oxime, thioketone, thioester, or thioamide. For example, in one such embodiment, EA₂ is halide, aldehyde, ketone, ester, carboxylic acid, amide, acyl chloride, trifluoromethyl, nitrile, sulfonic acid, ammonia, or an amine. For example, in this embodiment, EA₂ may be selected from the group consisting of —X, —C(O)H, —C(O)R, —C(O)OR, —C(O)OH, —C(O)X, —C(O)NRₐR_{b}, —C(X)₃, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen. By way of further example, in one such embodiment EA₂ may be selected from the group consisting of —C(O)H, —C(O)R, —C(O)OR, —C(O)OH, —C(O)X, —C(O)NRₐR_{b}, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen. By way of further example, in one such embodiment EA₂ may be selected from the group consisting of —C(O)H, —C(O)R, —C(O)OR, —C(O)OH, —C(O)X, —C(O)NRₐR_{b}, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen. By way of further example, in one such embodiment EA₂ may be selected from the group consisting of —C(O)H, —C(O)R, —C(O)OR, —C(O)OH, —C(O)X, and —C(O)NRₐR_{b}, where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen.

In accordance with one preferred embodiment, the diazo reagent corresponds to formula D-3

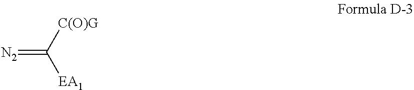

where G is hydrogen, hydroxy, alkoxy, hydrocarbyl, substituted hydrocarbyl, halogen or amino, and EA₁ is hydroxy, alkoxy, mercapto, halogen, carbonyl, sulfonyl, nitrile, quaternary amine, nitro, trihalomethyl, imine, amidine, oxime, thioketone, thioester, or thioamide. For example, in one such embodiment, G is hydrogen, hydroxy, alkoxy, hydrocarbyl, substituted hydrocarbyl, halogen or amino, and EA₁ is halide, aldehyde, ketone, ester, carboxylic acid, amide, acyl chloride, trifluoromethyl, nitrile, sulfonic acid, ammonia, or an amine. For example, in one such embodiment, G is hydrogen, hydroxy, alkoxy, hydrocarbyl, substituted hydrocarbyl, halogen or amino, and EA₁ is selected from the group consisting of —X, —C(O)H, —C(O)R, —C(O)OR, —C(O)OH, —C(O)X, —C(O)NRₐR_{b}, —C(X)₃, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl or heterocyclo, and X is halogen. By way of further example, in one such embodiment, G is hydrogen, hydroxy, alkoxy, hydrocarbyl, substituted hydrocarbyl, halogen or amino, and EA₁ is selected from the group consisting of —C(O)H, —C(O)R, —C(O)OR, —C(O)OH, —C(O)X, —C(O)NRₐR_{b}, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl or heterocyclo, and X is halogen. By way of further example, in one such embodiment, G is hydrogen, hydroxy, alkoxy, hydrocarbyl, substituted hydrocarbyl, halogen or amino, and EA₁ is selected from the group consisting of —CN, —SO₃H, —N⁺H₃, and —N⁺(R)₃ where R is hydrocarbyl, substituted hydrocarbyl or heterocyclo, and X is halogen. By way of further example, in one such embodiment G is hydrogen, hydroxy, alkoxy, hydrocarbyl, substituted hydrocarbyl, halogen or amino and EA₁ is selected from the group consisting of —C(O)H, —C(O)R, —C(O)OR, —C(O)OH, —C(O)X, and —C(O)NRₐR_{b}, where R is hydrocarbyl, substituted hydrocarbyl or heterocyclo, Rₐ and R_{b} are independently hydrogen, hydrocarbyl, substituted hydrocarbyl or heterocyclo, and X is halogen.

In one embodiment, the diazo reagent corresponds to Formula D-4:

N₂C(CN)C(O)OR₁₀ (Formula D-4)

where R₁₀ is hydrogen, hydrocarbyl, substituted hydrocarbyl or heterocyclo. For example, in one such embodiment, the diazo reagent has the formula N₂C(CN)C(O)OR₁₀ where R₁₀ is alkyl, aryl or alkaryl, more preferably lower alkyl or aryl. Other exemplary α-cyanodiazoacetates have the formula N₂C(CN)C(O)OR₁₀ where R₁₀ is lower alkyl such as methyl, ethyl, propyl or butyl.

In another embodiment, the diazo reagent corresponds to Formula D-5:

N₂C(CN)C(O)NRₐR_{b} (Formula D-5)

where Rₐ and R_{b} are independently hydrogen, hydrocarbyl, substituted hydrocarbyl, alkoxy or heterocyclo. For example, in one such embodiment, the diazo reagent corresponds to Formula D-5 and Rₐ and R_{b} are independently alkyl, aryl or alkaryl, more preferably lower alkyl or aryl. By way of further example, in one such embodiment, the diazo reagent corresponds to Formula D-5 and Rₐ and R_{b} are independently alkyl, aryl or alkaryl, more preferably lower alkyl or aryl. By way of further example, in one such embodiment, the diazo reagent corresponds to Formula D-5, Rₐ is alkyl, aryl or alkaryl, more preferably lower alkyl or aryl, and R_{b} is alkoxy, such as methoxy or ethoxy. Other exemplary α-cyanodiazoacetamides correspond to Formula D-5 wherein Rₐ and R_{b} are lower alkyl such as methyl, ethyl, propyl or butyl.

Metal Porphyrins

The metal porphyrin complex employed in the cyclopropenation reaction may be any of a wide range of porphyrins known in the art. Exemplary porphyrins are described in U.S. Patent Publication Nos. 2005/0124596 and 2006/0030718 and U.S. Pat. No. 6,951,935 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety.

In one embodiment, the metal porphyrin complex is a cobalt(II) porphyrin complex. In one particularly preferred embodiment, the cobalt porphyrin complex is a chiral porphyrin complex corresponding to the following structure:

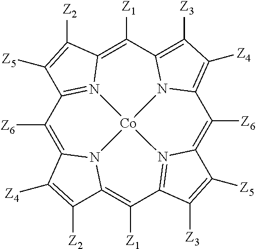

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, trifluoromethanesulfonate (OTf), haloaryl and haloalkyl. In a preferred embodiment, Z₂, Z₃, Z₄ and Z₅ are hydrogen, Z₁ is a substituted phenyl, Z₆ is substituted phenyl, and Z₁ and Z₆ are different. In one particularly preferred embodiment, Z₂, Z₃, Z₄ and Z₅ are hydrogen, Z₁ is substituted phenyl, Z₆ is substituted phenyl, Z₁ and Z₆ are different, and the porphyrin is a chiral porphyrin. In one even further preferred embodiment, Z₂, Z₃, Z₄ and Z₅ are hydrogen, Z₁ is substituted phenyl, Z₆ is substituted phenyl, Z₁ and Z₆ are different and the porphyrin has D₂-symmetry.

In a preferred embodiment, Z₁ is selected from the group consisting of

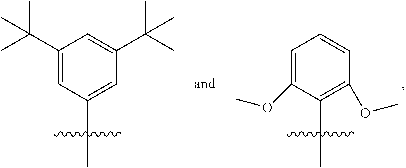

wherein

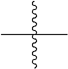

denotes the point of attachment to the porphyrin complex.

In a preferred embodiment, Z₆ is selected from the group consisting of

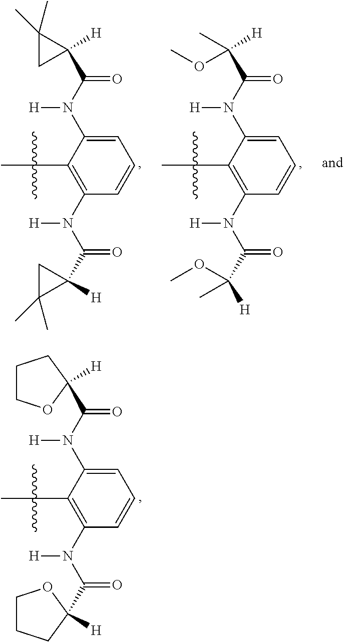

wherein

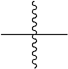

denotes the point of attachment to the porphyrin complex.

Exemplary cobalt (II) porphyrins include the following, designated [Co(P1)], [Co(P2)], [Co(P3)], [Co(P4)], [Co(P5)], [Co(P6)], and [Co(P7)]:

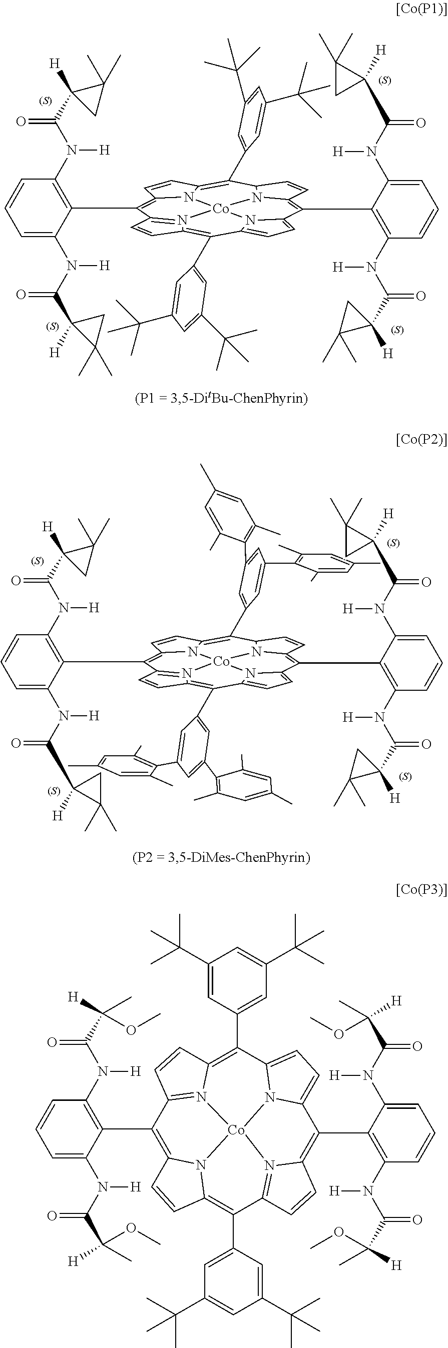

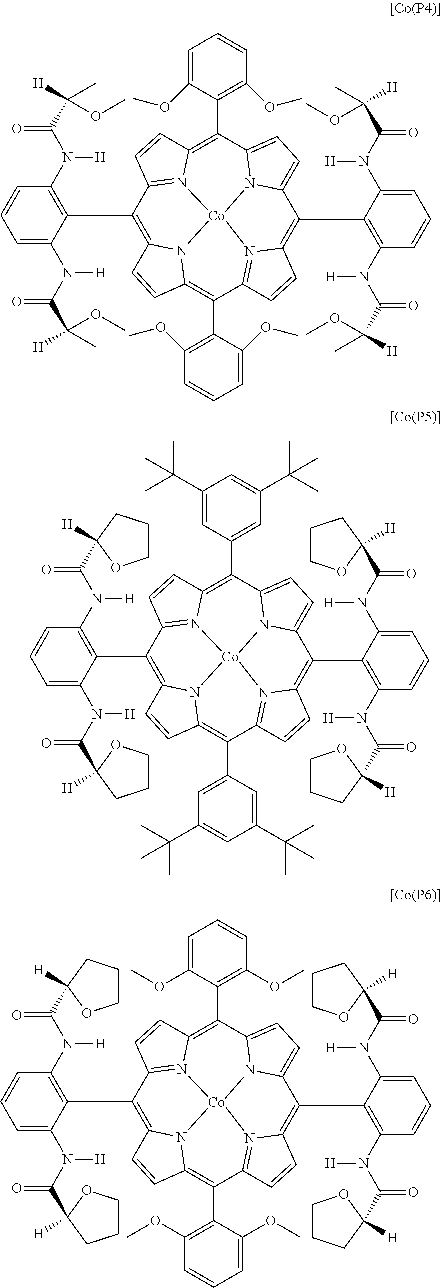

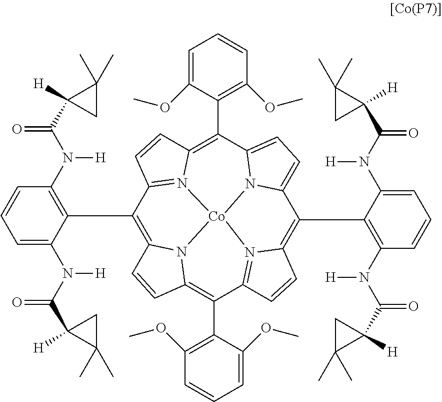

Cyclopropenation

Reaction Scheme 1 illustrates one embodiment of the cyclopropenation reaction of the present invention.

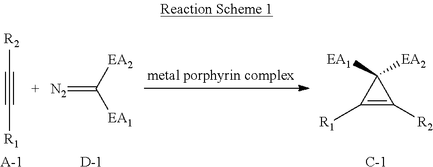

The substrate, alkyne A-1 is treated with an acceptor/acceptor-substituted diazo reagent D-1 in the presence of a metal porphyrin complex to produce cyclopropene C-1 wherein R₁ and R₂ are as previously defined in connection with Formula A-1, EA₁ and EA₂ are as previously defined in connection with Formula D-1 and the metal porphyrin complex is preferably a cobalt(II) porphyrin complex as previously defined.

In one preferred embodiment, the alkyne substrate corresponds to Formula A-2 and is treated with an acceptor/acceptor-substituted diazo reagent D-1 in the presence of a metal porphyrin complex to produce cyclopropene C-2 as depicted in Reaction Scheme 2

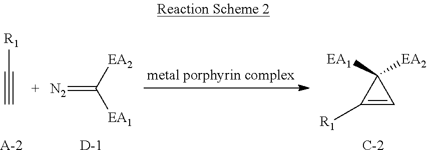

wherein R₁ is as previously defined in connection with Formula A-1, EA₁ and EA₂ are as previously defined in connection with Formula D-1 and the metal porphyrin complex is preferably a cobalt(II) porphyrin complex as previously defined.

In one preferred embodiment, the alkyne substrate corresponds to Formula A-2 and is treated with an acceptor/acceptor-substituted diazo reagent D-2 in the presence of a metal porphyrin complex to produce cyclopropene C-3 as depicted in Reaction Scheme 3

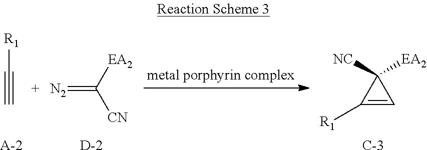

wherein R₁ is hydrocarbyl, substituted hydrocarbyl, heterocyclo, or an electron withdrawing group and EA₂ is hydroxy, alkoxy, mercapto, halogen, carbonyl, sulfonyl, nitrile, quaternary amine, nitro, trihalomethyl, imine, amidine, oxime, thioketone, thioester, or thioamide. For example, in one embodiment, R₁ may be alkyl or substituted alkyl. By way of further example, R₁ may be aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl; for example, R₁ may be —C(O)R, —C(O)OR, or —C(O)NRₐR_{b} wherein R, Rₐ and R_{b} are independently optionally substituted alkyl or optionally substituted aryl. By way of further example, R₁ may be heterocyclo; for example, R₁ may be optionally substituted furyl, thienyl, pyridyl or other five or six membered heterocyclo. In one such embodiment, EA₂ may be halide, aldehyde, ketone, ester, carboxylic acid, amide, acyl chloride, trifluoromethyl, nitrile, sulfonic acid, ammonia, or an amine. For example, in this embodiment, EA₂ may be selected from the group consisting of —X, —C(O)H, —C(O)R, —C(O)OR, —C(O)OH, —C(O)X, —C(O)NRₐR_{b}, —C(X)₃, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen. By way of further example, in one such embodiment EA₂ may be selected from the group consisting of —C(O)H, —C(O)R, —C(O)OR, —C(O)OH, —C(O)X, —C(O)NRₐR_{b}, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen. By way of further example, in one such embodiment EA₂ may be selected from the group consisting of —C(O)H, —C(O)R, —C(O)OR, —C(O)OH, —C(O)X, —C(O)NRₐR_{b}, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen. By way of further example, in one such embodiment EA₂ may be selected from the group consisting of —C(O)H, —C(O)R, —C(O)OR, —C(O)OH, —C(O)X, and —C(O)NRₐR_{b}, where R is hydrocarbyl, substituted hydrocarbyl or heterocyclo, Rₐ and R_{b} are independently hydrogen, hydrocarbyl, substituted hydrocarbyl, alkoxy or heterocyclo, and X is halogen.

In one preferred embodiment, the alkyne substrate corresponds to Formula A-2 and is treated with an acceptor/acceptor-substituted diazo reagent D-3 in the presence of a metal porphyrin complex to produce cyclopropene C-4 as depicted in Reaction Scheme 4

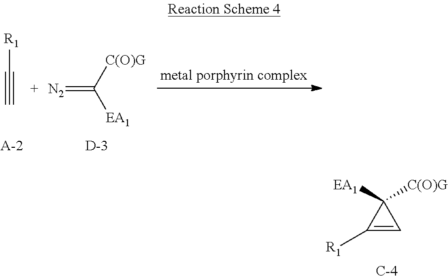

wherein R₁ is hydrocarbyl, substituted hydrocarbyl, heterocyclo, or an electron withdrawing group, G is hydrogen, hydroxy, alkoxy, hydrocarbyl, substituted hydrocarbyl, halogen or amino, and EA₁ is hydroxy, alkoxy, mercapto, halogen, carbonyl, sulfonyl, nitrile, quaternary amine, nitro, trihalomethyl, imine, amidine, oxime, thioketone, thioester, or thioamide and EA₂ is hydroxy, alkoxy, mercapto, halogen, carbonyl, sulfonyl, nitrile, quaternary amine, nitro, trihalomethyl, imine, amidine, oxime, thioketone, thioester, or thioamide. For example, in one embodiment, R₁ may be alkyl or substituted alkyl. By way of further example, R₁ may be aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl; for example, R₁ may be —C(O)R, —C(O)OR, or —C(O)NRₐR_{b} wherein R, Rₐ and R_{b} are independently optionally substituted alkyl or optionally substituted aryl. By way of further example, R₁ may be heterocyclo; for example, R₁ may be optionally substituted furyl, thienyl, pyridyl or other five or six membered heterocyclo. By way of further example, in one such embodiment, G is hydrogen, hydroxy, alkoxy, hydrocarbyl, substituted hydrocarbyl, halogen or amino, and EA₁ is halide, aldehyde, ketone, ester, carboxylic acid, amide, acyl chloride, trifluoromethyl, nitrile, sulfonic acid, ammonia, or an amine. For example, in one such embodiment, G is hydrogen, hydroxy, alkoxy, hydrocarbyl, substituted hydrocarbyl, halogen or amino, and EA₁ is selected from the group consisting of —X, —C(O)H, —C(O)R, —C(O)OR, —C(O)OH, —C(O)X, —C(O)NRₐR_{b}, —C(X)₃, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl or heterocyclo, and X is halogen. By way of further example, in one such embodiment, G is hydrogen, hydroxy, alkoxy, hydrocarbyl, substituted hydrocarbyl, halogen or amino, and EA₁ is selected from the group consisting of —C(O)H, —C(O)R, —C(O)OR, —C(O)OH, —C(O)X, —C(O)NRₐR_{b}, —CN, —SO₃H, —N⁺H₃, and —N⁺(R)₃ where R is hydrocarbyl, substituted hydrocarbyl or heterocyclo, Rₐ and R_{b} are independently hydrogen, hydrocarbyl, substituted hydrocarbyl or heterocyclo, and X is halogen. By way of further example, in one such embodiment, G is hydrogen, hydroxy, alkoxy, hydrocarbyl, substituted hydrocarbyl, halogen or amino, and EA₁ is selected from the group consisting of —CN, —SO₃H, —N⁺H₃, and —N⁺(R)₃ where R is hydrocarbyl, substituted hydrocarbyl or heterocyclo, and X is halogen. By way of further example, in one such embodiment G is hydrogen, hydroxy, alkoxy, hydrocarbyl, substituted hydrocarbyl, halogen or amino and EA₁ is selected from the group consisting of —C(O)H, —C(O)R, —C(O)OR, —C(O)OH, —C(O)X, and —C(O)NRₐR_{b}, where R is hydrocarbyl, substituted hydrocarbyl or heterocyclo, Rₐ and R_{b} are independently hydrogen, hydrocarbyl, substituted hydrocarbyl or heterocyclo, and X is halogen.

In one embodiment, the alkyne substrate corresponds to Formula A-2 and is treated with a diazo reagent corresponding to Formula D-4 in the presence of a metal porphyrin complex to produce cyclopropene C-5 as depicted in Reaction Scheme 5

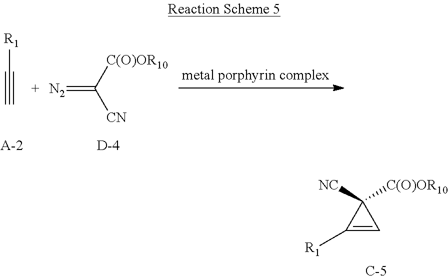

wherein R₁ is hydrocarbyl, substituted hydrocarbyl, heterocyclo, or an electron withdrawing group, and R₁₀ is hydrogen, hydrocarbyl, substituted hydrocarbyl or heterocyclo. For example, in one such embodiment, R₁ may be alkyl or substituted alkyl. By way of further example, R₁ may be aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl; for example, R₁ may be —C(O)R, —C(O)OR, or —C(O)NRₐR_{b} wherein R, Rₐ and R_{b} are independently optionally substituted alkyl or optionally substituted aryl. By way of further example, R₁ may be heterocyclo; for example, R₁ may be optionally substituted furyl, thienyl, pyridyl or other five or six membered heterocyclo. By way of further example, in one such embodiment, R₁₀ may be alkyl, aryl or alkaryl, more preferably lower alkyl or aryl.

In one embodiment, the alkyne substrate corresponds to Formula A-2 and is treated with a diazo reagent corresponding to Formula D-5 in the presence of a metal porphyrin complex to produce cyclopropene C-6 as depicted in Reaction Scheme 6

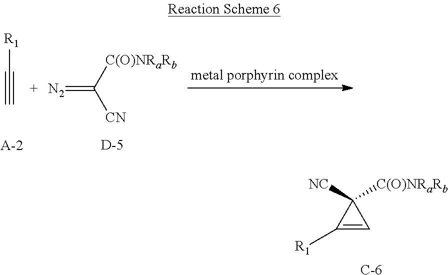

wherein R₁ is hydrocarbyl, substituted hydrocarbyl, heterocyclo, or an electron withdrawing group, and Rₐ and R_{b} are independently hydrogen, hydrocarbyl, substituted hydrocarbyl, alkoxy or heterocyclo. For example, in one such embodiment, R₁ may be alkyl or substituted alkyl. By way of further example, R₁ may be aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl; for example, R₁ may be —C(O)R, —C(O)OR, or —C(O)NRₐR_{b} wherein R, Rₐ and R_{b} are independently optionally substituted alkyl or optionally substituted aryl. By way of further example, R₁ may be heterocyclo; for example, R₁ may be optionally substituted furyl, thienyl, pyridyl or other five or six membered heterocyclo. By way of further example, in one such embodiment, Rₐ and R_{b} are independently hydrogen, hydrocarbyl, substituted hydrocarbyl, alkoxy or heterocyclo. For example, in one such embodiment, the diazo reagent corresponds to Formula D-5 and Rₐ and R_{b} are independently alkyl, aryl or alkaryl, more preferably lower alkyl or aryl. By way of further example, in one such embodiment, the diazo reagent corresponds to Formula D-5 and Rₐ and R_{b} are independently alkyl, aryl or alkaryl, more preferably lower alkyl or aryl. By way of further example, in one such embodiment, the diazo reagent corresponds to Formula D-5, Rₐ is alkyl, aryl or alkaryl, more preferably lower alkyl or aryl, and R_{b} is alkoxy, such as methoxy or ethoxy. Other exemplary α-cyanodiazoacetamides correspond to Formula D-5 wherein Rₐ and R_{b} are lower alkyl such as methyl, ethyl, propyl or butyl.

In a preferred embodiment, the metal porphyrin complex employed in Reaction Schemes 1-6 is a D₂-symmetric cobalt porphyrin catalyst corresponding to [Co(P1)] or [Co(P2)].

The cyclopropenation process illustrated by Reaction Schemes 1-6 presents a viable route to access densely functionalized cyclopropenes containing enantioenriched all-carbon quaternary stereogenic centers, which should find a range of applications as chiral building blocks for stereoselective organic syntheses through further functional group transformations. For instance, they can be transformed to highly functionalized cyclopropane derivatives by nucleophilic or electrophilic addition to the activated π-bonds as a result of the high ring strain.^{1,2} In particular, nucleophilic addition with soft nucleophiles would provide an entry to chiral hetero-substituted cyclopropanes, which would be difficult or impossible to access via direct asymmetric cyclopropanation of alkenes. The nucleophile may be, for example, a carbon nucleophile (e.g., alkyl metal halides found in the Grignard reaction, Blaise reaction, Reformatsky reaction, and Barbier reaction, organolithium reagents, and anions of a terminal alkyne, and enols), oxygen nucleophiles (e.g., water hydroxide, alcohols, and alkoxides), sulfur nucleophiles (e.g., thiols), and nitrogen nucleophiles (e.g., ammonia, azide, and amines). Alternatively, the cyclopropene may be derivatized by electrophilic addition with halogens or hydrogen halides, or by hydration.

As an initial effort toward this type of applications, we demonstrated that cyclopropene 3af could undergo highly diastereoselective addition reactions with thiol nucleophiles to furnish hetero-substituted cyclopropane derivatives (Table 4).¹⁶ For example, when 3af in 98% ee was treated with 1.5 equiv of n-propanethiol, the corresponding 1,1,2,3-tetra-substituted cyclopropane 4a could be isolated in 98% yield as a sole diastereomer in the same high optical purity (entry 1). The absolute configuration of the three continuous stereogenic centers in 4a was established to be [1S,2R,3S] by X-ray crystal structural analysis (see Supporting Information). Highly diastereoselective addition reactions of 3af could be similarly accomplished with isopropanethiol and tert-butylthiol, affording enantiopure thiolated cyclopropanes 4b and 4c, respectively, albeit in relatively lower yields due to the higher steric hindrance (entries 2 and 3).

### REFERENCES

(1) (a) Rubin, M.; Rubina, M.; Gevorgyan, V. Chem. Rev. 2007, 107, 3117. (b) Rubin, M.; Rubina, M.; Gevorgyan, V. Synthesis-Stuttgart 2006, 1221. (c) Fox, J. M.; Yan, N. Curr. Org. Chem. 2005, 9, 719. (d) Nakamura, M.; Isobe, H.; Nakamura, E. Chem. Rev. 2003, 103, 1295. (e) Carter, F. L.; Frampton, V. L. Chem. Rev. 1964, 64, 497. (f) Prosser, A. R.; Banning, J. E.; Rubina, M.; Rubin, M. Org. Lett. 2010, 12, 3968. (g) Banning, J. E.; Prosser, A. R.; Rubin, M. Org. Lett. 2010, 12, 1488. (h) Alnasleh, B. K.; Sherrill, W. M.; Rubina, M.; Banning, J.; Rubin, M. J. Am. Chem. Soc. 2009, 131, 6906. (i) Sherrill, W. M.; Rubin, M. J. Am. Chem. Soc. 2008, 130, 13804. (j) Alnasleh, B. K.; Sherrill, W. M.; Rubin, M. Org. Lett. 2008, 10, 3231. (k) Xie, X. C.; Yang, Z.; Fox, J. M. J. Org. Chem. 2010, 75, 3847. (l) Tarwade, V.; Liu, X. Z.; Yan, N.; Fox, J. M. J. Am. Chem. Soc. 2009, 131, 5382. (m) Yan, N.; Liu, X. Z.; Fox, J. M. J. Org. Chem. 2008, 73, 563. (n) Fisher, L. A.; Fox, J. M. J. Org. Chem. 2008, 73, 8474.
(2) Marek, I.; Simaan, S.; Masarwa, A. Angew. Chem. Int. Edit. 2007, 46, 7364.
(3)(a) Protopopova, M. N.; Doyle, M. P.; Müller, P.; Ene, D. J. Am. Chem. Soc. 1992, 114, 2755. (b) Doyle, M. P.; Protopopova, M.; Müller, P.; Ene, D.; Shapiro, E. A. J. Am. Chem. Soc. 1994, 116, 8492. (c) Imogai, H.; Bernardinelli, G.; Granicher, C.; Moran, M.; Rossier, J. C.; Müller, P. Helv. Chim. Acta 1998, 81, 1754. (d) Müller, P.; Imogai, H. Tetrahedron-Asymmetry 1998, 9, 4419. (e) Lou, Y.; Horikawa, M.; Kloster, R. A.; Hawryluk, N. A.; Corey, E. J. J. Am. Chem. Soc. 2004, 126, 8916. (f) Lou, Y.; Remarchuk, T. P.; Corey, E. J. J. Am. Chem. Soc. 2005, 127, 14223.
(4) For asymmetric intramolecular cyclopropenation with diazoacetates, see: (a) Doyle, M. P.; Ene, D. G.; Forbes, D.C.; Pillow, T. H. Chem. Commun. 1999, 1691. (b) Doyle, M. P.; Ene, D. G.; Peterson, C. S.; Lynch, V. Angew. Chem. Int. Edit. 1999, 38, 700. (c) Doyle, M. P.; Hu, W. H. Tetrahedron Lett. 2000, 41, 6265. (d) Doyle, M. P.; Hu, W. H. Synlett 2001, 1364. (e) Doyle, M. P.; Weathers, T. M.; Wang, Y. H. Adv. Synth. Catal. 2006, 348, 2403.
(5) Weatherhead-Kloster, R. A.; Corey, E. J. Org. Lett. 2006, 8, 171.
(6)(a) Davies, H. M. L.; Lee, G. H. Org. Lett. 2004, 6, 1233. (b) Chuprakov, S.; Gevorgyan, V. Org. Lett. 2007, 9, 4463.
(7) Briones, J. F.; Hansen, J.; Hardcastle, K. I.; Autschbach, J.; Davies, H. M. L. J. Am. Chem. Soc. 2010, 132, 17211.
(8) Panne, P.; Fox, J. M. J. Am. Chem. Soc. 2007, 129, 22. Although the issue of enantioselectivity was not addressed, it was shown that Rh₂ complexes of sterically demanding carboxylate ligands could effectively catalyze cyclopropenation with α-alkyldiazoacetates possessing β-hydrogens, a class of donor/acceptor-substituted diazo reagents that are challenging for metal-mediated carbene transfer reactions due to competitive β-elimination.
(9) For a recent non-asymmetric synthesis of trifluoromethyl-substituted cyclopropenes via Rh₂-catalyzed cyclopropenation of alkynes, see: Morandi, B.; Carreira, E. M. Angew. Chem. Int. Edit. 2010, 49, 4294.
(10) Katsuki and coworkers reported a Ir(salen)-catalyzed asymmetric cyclorpropenation system that is effective with a cyclic α-cyanodiazoacetamide in addition to several donor/acceptor-substituted diazo reagents, see: Uehara, M.; Suematsu, H.; Yasutomi, Y.; Katsuki, T. J. Am. Chem. Soc. 2010, Article ASAP; DOI: 10.1021/ja1089217.
(11)(a) Doyle, M. P. Angew. Chem. Int. Edit. 2009, 48, 850. (b) Huang, L. Y.; Chen, Y.; Gao, G. Y.; Zhang, X. P. J. Org. Chem. 2003, 68, 8179. (c) Penoni, A.; Wanke, R.; Tollari, S.; Gallo, E.; Musella, D.; Ragaini, F.; Demartin, F.; Cenini, S. Eur. J. Inorg. Chem. 2003, 1452.
(12) Chen, Y.; Fields, K. B.; Zhang, X. P. J. Am. Chem. Soc. 2004, 126, 14718.
(13)(a) Chen, Y.; Zhang, X. P. J. Org. Chem. 2007, 72, 5931. (b) Chen, Y.; Ruppel, J. V.; Zhang, X. P. J. Am. Chem. Soc. 2007, 129, 12074. (c) Zhu, S. F.; Ruppel, J. V.; Lu, H. J.; Wojtas, L.; Zhang, X. P. J. Am. Chem. Soc. 2008, 130, 5042. (d) Zhu, S. F.; Perman, J. A.; Zhang, X. P. Angew. Chem. Int. Ed. 2008, 47, 8460. (e) Fantauzzi, S.; Gallo, E.; Rose, E.; Raoul, N.; Caselli, A.; Issa, S.; Ragaini, F.; Cenini, S. Organometallics 2008, 27, 6143. (f) Ruppel, J. V.; Gauthier, T. J.; Snyder, N. L.; Perman, J. A.; Zhang, X. P. Org. Lett. 2009, 11, 2273. (g) Zhu, S. F.; Xu, X.; Perman, J. A.; Zhang, X. P. J. Am. Chem. Soc. 2010, 132, 12796.
(14) Dzik, W. I.; Xu, X.; Zhang, X. P.; Reek, J. N. H.; de Bruin, B. J. Am. Chem. Soc. 2010, 132, 10891.
(15)(a) Balasubramaniam, S.; Aidhen, I. S. Synthesis-Stuttgart 2008, 3707. (b) Khlestkin, V. K.; Mazhukin, D. G. Curr. Org. Chem. 2003, 7, 967.
(16)(a) Martinez-Grau, A.; Blasco, J. M.; Ferritto, R.; Espinosa, J. F.; Mantecon, S.; Vaquero, J. J. Arkivoc 2005, 394. (b) Shapiro, E. A.; Kalinin, A. V.; Nefedov, O. M. Org. Prep. Proced. Int. 1992, 24, 517.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

Initial experiments were focused on the evaluation of ligand and solvent effects on cyclopropenation of phenylacetylene (1a) with α-cyano(N,N-dimethyl)diazoacetamide (2a) by Co(Por) under conditions that were deemed most practical: 1 mol % catalyst loading; room temperature; stoichiometric ratio of reactants; and one-time protocol without slow addition (Table 1). While the ineffectiveness of [Co(TPP)] for the reaction might be expected due to the absence of the H-bonding donor amide units (entry 1), we were somewhat surprised by the inferior performance of [Co(P1)], which has been previously shown to be highly effective for various cyclopropanation reactions.^{12,13} This result indicated different requirements of catalyst environment for the two carbene transfer processes and prompted us to develop new catalysts by taking advantage of the modular design and tunability of the D₂-symmetric chiral porphyrin system.^{11a,12} To this end, replacement of the aliphatic t-butyl substituent in P1 with aromatic mesityl group led to the design and synthesis of new D₂-symmetric chiral porphyrin 3,5-diMes-ChenPhyrin (P2). Gratifyingly, [Co(P2)] was found to be a highly effective catalyst for the reaction, leading to the formation of the desired 1,1-cyclopropeneamidonitrile 3aa in 95% yield and 82% ee (entry 3). During the process of optimizing the reaction conditions, trifluorotoluene was shown to be the solvent of choice and performed significantly better than other solvents screened (entries 4-9).

TABLE 1
Reaction Conditions for Cyclopropenation of Phenylacetylene
with α-Cyano(N,N-dimethyl)diazoacetamide by Cobalt(II) Porphyrins.^{a}

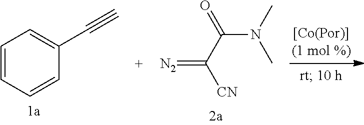

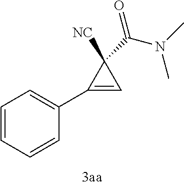 [SMILES: CN(C)C(=O)[C@]1(C#N)C=C1c1ccccc1]

| entry | [Co(Por)]^{b} | solvent | yield (%)^{c} | ee (%)^{d} |
|---|---|---|---|---|
| 1 | [Co(TPP)] | PhCF₃ | <5 | nd^{e} |
| 2 | [Co(P1)] | PhCF₃ | 10 | 71 |
| 3 | [Co(P2)] | PhCF₃ | 95 | 82 |
| 4 | [Co(P2)] | PhCl | 40 | nd^{e} |
| 5 | [Co(P2)] | PhF | 38 | nd^{e} |
| 6 | [Co(P2)] | PhMe | 26 | nd^{e} |
| 7 | [Co(P2)] | PhH | 45 | nd^{e} |
| 8 | [Co(P2)] | CH₂Cl | <5 | nd^{e} |
| 9 | [Co(P2)] | CCl₄ | 70 | 77 |

^{a}Reactions were carried out at room temperature for 10 h in one-time fashion without slow addition of the diazo reagent using 1 mol % [Co(Por)] under N₂ with 1.0 equiv of α-cyanodiazo(N,N-dimethyl)acetamide and 1.5 equiv of phenylacetylene. Concentration: 0.10 mmol diazo/mL.
^{b}See FIG.1 for structure.
^{c}Isolated yields.
^{d}Enantiomeric excess determined by chiral HPLC.
^{e}Not determined

The [Co(P2)]-based metalloradical cyclopropenation system was found to be applicable for different acceptor/acceptor-substituted diazo reagents under the similar practical conditions (Table 2). Like the N,N-dimethyl diazo 2a (entry 1), N,N-diethyl analog 2b could also function as effective carbene source for Co(II)-catalyzed enantioselective cyclopropenation of 1a (entry 2). Notably, the catalytic system went equally well with N-methoxy-N-methyl α-cyanodiazoacetamide 2c, providing the corresponding chiral cyclopropenyl Weinreb amide 3ac (entry 3), which can serve as a potential synthon for preparation of chiral cyclopropenyl cyanoaldehyde and cyanoketone derivatives.¹⁵ In addition to the tertiary analogs, secondary α-cyanodiazoacetamides could also be productively used as exemplified with N-isopropyl α-cyanodiazoacetamide 2d, forming the desired cyclopropene 3ad in 96% yield and 96% ee (entry 4). Besides α-cyanodiazoacetamides, [Co(P2)] was shown to enable cyclopropenation with α-cyanodiazoacetates as well. For example, both ethyl and t-butyl α-cyanodiazoacetates (2e and 2f) could be effectively utilized to cylopropenate 1a to form 1,1-cyclopropeneesternitrile 3ae and 3af, respectively, in good yields and excellent enantioselectivities (entries 5 and 6). The absolute configuration of the all-carbon quaternary stereogenic center in 3af was established as [R] by X-ray crystal structural analysis (see Supporting Information).

TABLE 2
[Co(P2)]-Catalyzed Enantioselective Cyclopropenation of
Phenylacetylene with Acceptor/Acceptor-Substituted Diazo Reagents.^{a}

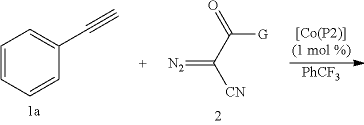

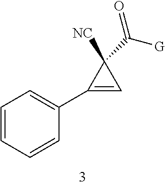

| | | | yield | ee |
|---|---|---|---|---|
| entry | diazo | cyclopropene | (%)^{b} | (%)^{c} |
| 1^{d} | 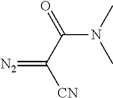 2a | 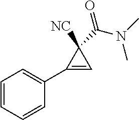 [SMILES: CN(C)C(=O)[C@]1(C#N)C=C1c1ccccc1] 3aa | 95 | 82 |
| 2^{e} | 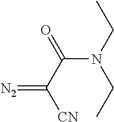 2b | 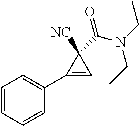 [SMILES: CCN(CC)C(=O)[C@]1(C#N)C=C1c1ccccc1] 3ab | 92 | 80 |
| 3^{d} | 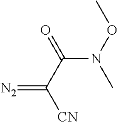 2c | 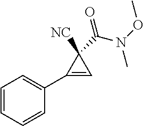 [SMILES: CON(C)C(=O)[C@]1(C#N)C=C1c1ccccc1] 3ac | 89 | 83 |
| 4^{d} | 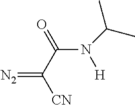 2d | 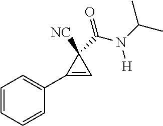 [SMILES: CC(C)NC(=O)[C@]1(C#N)C=C1c1ccccc1] 3ad | 96 | 96 |
| 5^{d} | 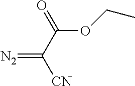 2e | 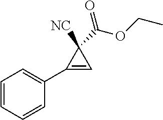 [SMILES: CCOC(=O)[C@]1(C#N)C=C1c1ccccc1] 3ae | 77 | 93 |
| 6^{d} | 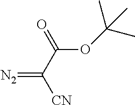 2f | 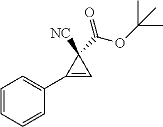 [SMILES: CC(C)(C)OC(=O)[C@]1(C#N)C=C1c1ccccc1] 3af | 76 | 98^{f} |

^{a}Reactions were carried out in one-time fashion without slow addition of the diazo reagent using 1 mol % [Co(P2)] under N₂ with 1.0 equiv of diazo reagent and 1.5 equiv of phenylacetylene. Concentration: 0.10 mmol diazo/mL PhCF₃.
^{b}Isolated yields.
^{c}Enantiomeric excess determined by chiral HPLC.
^{d} At room temperature for 12 h.
^{e}At 40° C. for 24 h.
^{f}[R] absolute configuration determined by anomalous-dispersion effects in X-ray diffraction measurements on the crystal.

The [Co(P2)]-catalyzed asymmetric cyclopropenation could be successfully expanded for a wide range of terminal aromatic and related conjugated alkynes in combination with various acceptor/acceptor-substituted diazo reagents (Table 3). For example, using N-isopropyl diazo 2d as the carbene source, enantioselective cyclopropenation reactions of phenylacetylenes substituted with alkyl groups at different positions proceeded equally well as phenylacetylene (entries 1 and 2). In addition, various halogenated phenylacetylenes could be enantioselectively cyclopropenated with both α-cyanodiazoacetamides and -acetates (entries 3-8). Among the chiral cyclopropene products, the absolute configuration of the all-carbon quaternary stereogenic center in 3df (entry 5) was established to be [R] by X-ray crystal structural analysis (see Supporting Information). Chiral cyclopropene derivatives from reactions of aromatic alkynes containing both electron-withdrawing and -donating groups could also be obtained in good yields and high enantioselectivities (entries 9 and 10). The Co(II)-catalyzed reaction could be extended for non-aromatic conjugated alkynes as demonstrated with cyclopropenation reaction of cyclohexenylethyne with diazo 2d for the high-yielding formation of enantioenriched 1,1-cyclopropeneamidonitrile 3jd (entry 11).

TABLE 3
[Co(P2)]-Catalyzed Asymmetric Cyclopropenation of Different
Combinations of Aryl/Vinyl Alkynes and A/A-Type Diazo Reagents.^{a}

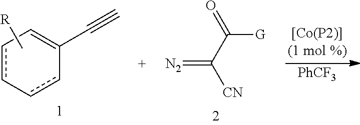

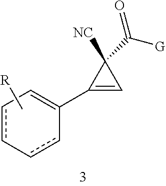

| | |
|---|---|
| 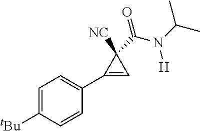 [SMILES: CC(C)NC(=O)[C@]1(C#N)C=C1c1ccc(C(C)(C)C)cc1] entry 1: 3bd^{b} 80% yield; 90% ee | 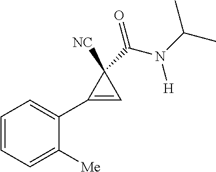 [SMILES: Cc1ccccc1C1=C[C@@]1(C#N)C(=O)NC(C)C] entry 2: 3cd^{b} 93% yield; 92% ee |
| 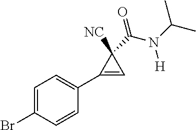 [SMILES: CC(C)NC(=O)[C@]1(C#N)C=C1c1ccc(Br)cc1] entry 3: 3dd^{b} 83% yield; 97% ee | 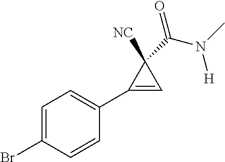 [SMILES: CNC(=O)[C@]1(C#N)C=C1c1ccc(Br)cc1] entry 4: 3da^{b} 97% yield; 83% ee |
| 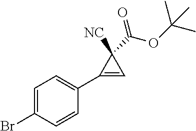 [SMILES: CC(C)(C)OC(=O)[C@]1(C#N)C=C1c1ccc(Br)cc1] entry 5: 3df^{c, d} 59% yield; 99% ee | 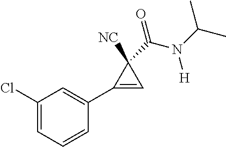 [SMILES: CC(C)NC(=O)[C@]1(C#N)C=C1c1cccc(Cl)c1] entry 6: 3ed^{b} 75% yield; 98% ee |
| 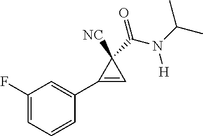 [SMILES: CC(C)NC(=O)[C@]1(C#N)C=C1c1cccc(F)c1] entry 7: 3fd^{b} 50% yield; 98% ee | 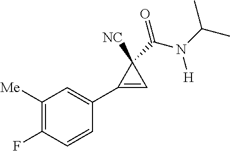 [SMILES: Cc1cc(C2=C[C@@]2(C#N)C(=O)NC(C)C)ccc1F] entry 8: 3gd^{b} 80% yield; 94% ee |
| 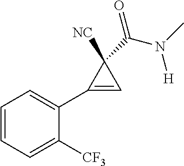 [SMILES: CNC(=O)[C@]1(C#N)C=C1c1ccccc1C(F)(F)F] entry 9: 3ha^{c} 84% yield; 91% ee | 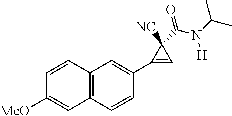 [SMILES: COc1ccc2cc(C3=C[C@@]3(C#N)C(=O)NC(C)C)ccc2c1] entry 10: 3id^{b} 71% yield; 88% ee |
| 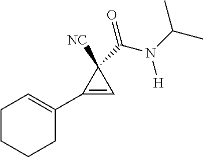 [SMILES: CC(C)NC(=O)[C@]1(C#N)C=C1C1=CCCCC1] entry 11: 3jd^{b} 95% yield; 89% ee | 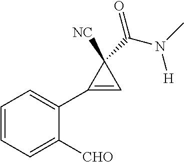 [SMILES: CNC(=O)[C@]1(C#N)C=C1c1ccccc1C=O] entry 12: 3ka^{c} 85% yield; 82% ee |
| 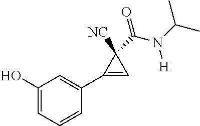 [SMILES: CC(C)NC(=O)[C@]1(C#N)C=C1c1cccc(O)c1] entry 13: 3ld^{b} 62% yield; 92% ee | 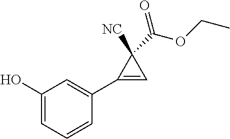 [SMILES: CCOC(=O)[C@]1(C#N)C=C1c1cccc(O)c1] entry 14: 3le^{b} 52% yield; 88% ee |

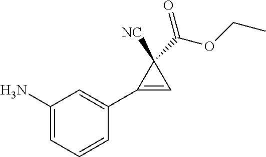 [SMILES: CCOC(=O)[C@]1(C#N)C=C1c1cccc(N)c1]

entry 15: 3me^{b} 42% yield; 95% ee
^{a}Reactions were carried out in one-time fashion without slow addition of the diazo reagent using 1 mol % [Co(P2)] under N₂ with 1.0 equiv of diazo reagent and 1.5 equiv of phenylacetylene. Concentration: 0.10 mmol diazo/mL PhCF₃; Isolated yields; Enantiomeric excess determined by chiral HPLC.
^{b}At room temperature for 12 h.
^{c}At 40° C. for 24 h.
^{d}[R] absolute configuration; see footnote f of Table 2.

Consistent with the proposed radical mechanism of non-electrophilic carbene radical intermediate,¹⁴ the Co(II)-catalyzed carbene transfer process was found to tolerate functional groups that would otherwise undergo ylide-type chemistry associated with electrophilic metallocarbenes. For example, the aldehyde functionality could be tolerated without complication from potential ylide-mediated epoxidation (entry 12). The functional group tolerability of [Co(P2)]-catalyzed asymmetric cyclopropenation were further highlighted by the reactions of phenylacetylene derivatives containing hydroxyl and amino substituents (entries 13-15). In all the cases, no O—H or N—H insertion products were observed.

The above demonstrated cyclopropenation process via [Co(P2)]-based metalloradical catalysis presents a viable route to access densely functionalized cyclopropenes containing enantioenriched all-carbon quaternary stereogenic centers, which should find a range of applications as chiral building blocks for stereoselective organic syntheses through further functional group transformations. For instance, they can be transformed to highly functionalized cyclopropane derivatives by nucleophilic or electrophilic addition to the activated π-bonds as a result of the high ring strain.^{1,2} In particular, nucleophilic addition with soft nucleophiles would provide an entry to chiral hetero-substituted cyclopropanes, which would be difficult or impossible to access via direct asymmetric cyclopropanation of alkenes. As an initial effort toward this type of applications, we demonstrated that cyclopropene 3af could undergo highly diastereoselective addition reactions with thiol nucleophiles to furnish hetero-substituted cyclopropane derivatives (Table 4).¹⁶ For example, when 3af in 98% ee was treated with 1.5 equiv of n-propanethiol, the corresponding 1,1,2,3-tetra-substituted cyclopropane 4a could be isolated in 98% yield as a sole diastereomer in the same high optical purity (entry 1). The absolute configuration of the three continuous stereogenic centers in 4a was established to be [1S,2R,3S] by X-ray crystal structural analysis (see Supporting Information). Highly diastereoselective addition reactions of 3af could be similarly accomplished with isopropanethiol and tert-butylthiol, affording enantiopure thiolated cyclopropanes 4b and 4c, respectively, albeit in relatively lower yields due to the higher steric hindrance (entries 2 and 3).

TABLE 4
Diastereoselective Thiol Addition to Cyclopropenes.^{a}

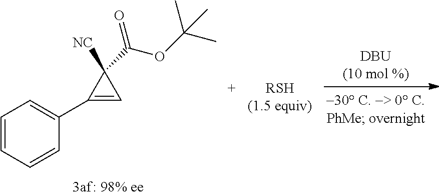 [SMILES: CC(C)(C)OC(=O)[C@]1(C#N)C=C1c1ccccc1]

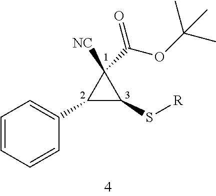

| | | | yield | ee |
|---|---|---|---|---|
| entry | thiol | product | (%)^{b} | (%)^{c} |
| 1 | 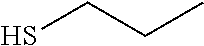 | 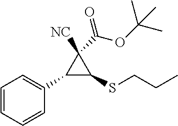 [SMILES: CCCS[C@H]1[C@H](c2ccccc2)[C@]1(C#N)C(=O)OC(C)(C)C] 4a | 98 | 98^{d} |
| 2 | 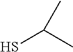 | 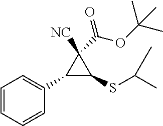 [SMILES: CC(C)S[C@H]1[C@H](c2ccccc2)[C@]1(C#N)C(=O)OC(C)(C)C] 4b | 88 | 98 |
| 3^{e} | 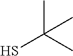 | 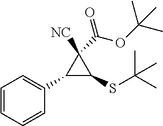 [SMILES: CC(C)(C)OC(=O)[C@]1(C#N)[C@@H](SC(C)(C)C)[C@@H]1c1ccccc1] 4c | 54 | 98 |

^{a}Reactions were carried out overnight in toluene using 10 mol % DBU with 1.0 equiv of cyclopropene 3af (98% ee) and 1.5 equiv of thiol. The reaction temperature was initially at −30° C. and gradually warmed to room temperature.
^{b}Isolated yields of single diastereomers.
^{c}Enantiomeric excess determined by chiral HPLC.
^{d}[1S,2R,3S] absolute configuration determined by anomalous-dispersion effects in X-ray diffraction measurements on crystal.
^{e}100 mol % DBU; room temperature; 48 h .

Furthermore, we showed that the thiolated cyclopropane 4a could be oxidatively converted to cyclopropyl sulfone 5a in a high yield without loss of its optical purity upon simple treatment with m-CPBA at room temperature (eq 1).

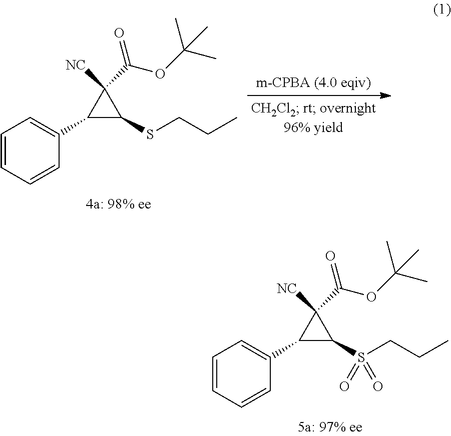 [SMILES: CCCS[C@H]1[C@H](c2ccccc2)[C@]1(C#N)C(=O)OC(C)(C)C ; CCCS(=O)(=O)[C@H]1[C@H](c2ccccc2)[C@]1(C#N)C(=O)OC(C)(C)C]

In summary, we have developed a highly enantioselective process based on the new metalloradical catalyst [Co(P2)] for cyclopropenation of aryl/vinyl alkynes with both α-cyanodiazoacetamides and α-cyanodiazoacetates. It represents the first successful applications of these two types of acceptor-acceptor-substituted diazo reagents for asymmetric cyclopropenation,¹⁰ providing a practical method for the preparation of multi-functionalized cyclopropenes bearing enantioenriched all-carbon quaternary stereogenic centers that may serve as useful chiral synthons for stereoselective synthesis (Scheme 1). Among several salient features, the Co(II)-based system enjoys an unusual degree of functional group tolerance, which is believed to have close relevance to the radical pathway of Co(II)-based metalloradical catalysis.¹⁴

General Considerations

All reactions were carried out under a nitrogen atmosphere in oven-dried glassware following standard Schlenk techniques. α,α,α-Trifluorotoluene (Anhydrous, ≧99%) was used directly from Sigma-Aldrich Chemical Co. All cross-coupling reactions were carried out under a nitrogen atmosphere in oven-dried glassware following standard Schlenk techniques. Tetrahydrofuran (THF) and toluene were distilled under nitrogen from sodium benzophenone ketyl. Chiral amides were purchased from Aldrich Chemical Co. and Acros Organics, used without further purification. Anhydrous cobalt(II) chloride, palladium(II) acetate, and 9,9-dimethyl-4,5-bis(diphenylphosphino)xanthene (Xantphos) were purchased from Strem Chemical Co. Cesium carbonate was obtained as a gift from Chemetall Chemical Products, Inc. Thin layer chromatography was performed on Merck TLC plates (silica gel 60 F254). Flash column chromatography was performed with ICN silica gel (60 Å, 230-400 mesh, 32-63 μm). Proton and carbon nuclear magnetic resonance spectra (¹H NMR and ¹³C NMR) were recorded on a Bruker 250-MHz instrument and referenced with respect to internal TMS standard. HPLC measurements were carried out on a Shimadzu HPLC system with Whelk-O1, Chiralcel OD-H, OJ-H, and AD-H columns. Infared spectra were measured with a Nicolet Avatar 320 spectrometer with a Smart Miracle accessory. HRMS data was obtained on an Agilent 1100 LC/MS ESI/TOF mass spectrometer with electrospray ionization.

Preparation of TfN₃ hexane solution:¹

A solution of sodium azide (10 g, 150 mmol) and tetrabutylammonium hydrogen sulfate, Bu₄NHSO₄, (0.56 g, 1.64 mmol) in distilled water (30 mL) was cooled to 0° C. A solution of triflic anhydride (8.46 g, 5.0 mL, 30 mmol) in hexane (25 mL) was then slowly added, and the resulting clear solution was stirred for an additional 1 h at 0° C. The reaction mixture was then extracted with hexane (25 mL), and the organic layer was dried over sodium hydroxide pellets and decanted. The hexane solution of triflyl azide was used immediately in the subsequent reaction. Alternatively, it could be stored at −15° C. for several weeks without significant decomposition. The concentration of the azide was estimated based on the total volume of the solution and assuming a quantitative conversion based on the amount of triflic anhydride used.

Preparation of TfN₃ hexane solution:¹

A solution of sodium azide (10 g, 150 mmol) and tetrabutylammonium hydrogen sulfate, Bu₄NHSO₄, (0.56 g, 1.64 mmol) in distilled water (30 mL) was cooled to 0° C. A solution of triflic anhydride (8.46 g, 5.0 mL, 30 mmol) in hexane (25 mL) was then slowly added, and the resulting clear solution was stirred for an additional 1 h at 0° C. The reaction mixture was then extracted with hexane (25 mL), and the organic layer was dried over sodium hydroxide pellets and decanted. The hexane solution of triflyl azide was used immediately in the subsequent reaction. Alternatively, it could be stored at −15° C. for several weeks without significant decomposition. The concentration of the azide was estimated based on the total volume of the solution and assuming a quantitative conversion based on the amount of triflic anhydride used.

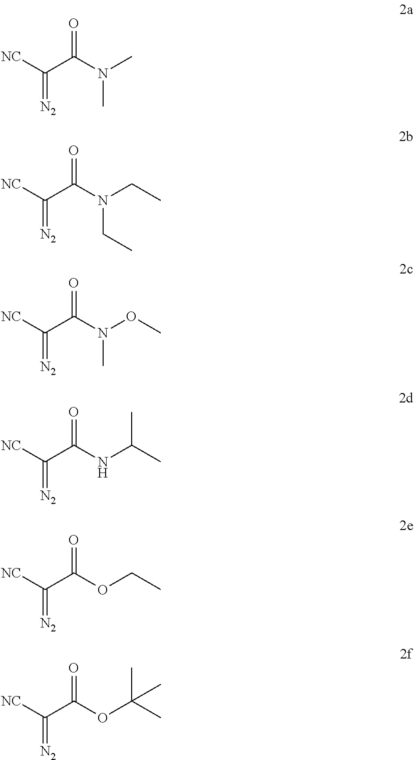

2-cyano-N-isopropylacetamide⁴, 2-cyano-N-methyl-N-methoxyacetamide⁵ were synthesized according to the literature. Using these precursors, 2-cyano-2-diazo-N-iso-propylacetamide (2d), 2-cyano-2-diazo-N-methyl-N-methoxyacetamide (2c) were synthesized by the following diazo transfer procedure: to a stirred solution of the 2-cyanodiazoacetate or 2-cyanodiazoacetamide (20 mmol) in acetonitrile (10 mL) was added the above triflyl azide solution (50 mL, 30 mmol) in hexane. Pyridine (4 mL, 53 mmol) was then added dropwise (over ca. 5 min). The reaction mixture was stirred at room temperature until starting material, after which the solvent was blown away by the air flow (Note: DON'T USE ROTARY EVAPORATOR AS IT MAY CAUSE POTENTIAL EXPLOSION). Purification of the crude residue by flash chromatography on silica gel afforded the pure products:

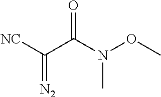

### 2-cyano-2-diazo-N-methyl-N-methoxyacetamide (2c)

75% yield. ¹H NMR (250 MHz, CDCl₃): δ 3.726 (s, 3H), 3.23 (s, 3H); ¹³C NMR (62.5 MHz, CDCl₃): 160.736, 108.713, 62.163, 53.475, 33.900. IR (neat, cm⁻¹): 2219.52, 2127.80, 1651.38, 1532.36, 1268.47, 612.34.

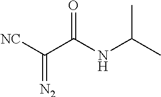

### 2-cyano-2-diazo-N-iso-propylacetamide (2d)

21% yield. ¹H NMR (250 MHz, CDCl₃): δ 4.20-3.99 (m, 1H), 5.49-5.25 (m, 1H), 1.16 (t, J=7.65 Hz, 6H); ¹³C NMR (62.5 MHz, CDCl₃): 157.873, 109.177, 43.397, 22.653. IR (neat, cm⁻¹): 2221.55, 2124.75, 1644.64, 1383.33, 1209.73, 983.03, 708.49.

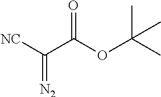

### tert-Butyl 2-cyano-2-diazoacetate (2f)

68% yield. ¹H NMR (250 MHz, CDCl₃): δ 1.46 (s, 9H); ¹³C NMR (62.5 MHz, CDCl₃): 160.125, 107.869, 85.495, 28.073. IR (neat, cm⁻¹): 2228.02, 2129.71, 1708.98, 1304.46, 1125.71, 838.96, 739.66.

Catalyst Synthesis:

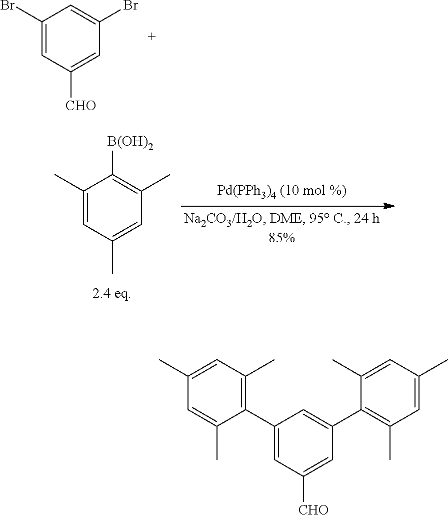

### 3,5-di(2,4,6-trimethylphenyl)benzaldehyde

A mixture of 3,5-dibromobenzaldehyde (5 mmol), 2,4,6-trimethylphenylboronic acid (12 mmol), Pd(PPh₃)₄ (0.5 mmol), Na₂CO₃ (20 mmol) in 50 ml DME and 10 ml H₂O was stirred under N₂ at 95 for 24 h. After the mixture was washed by water, extracted by ether and evaporated, the residue was purified by flash column chromatography (hexane/ethyl acetate) to afford 3,5-di(2,4,6-trimethylphenyl)benzaldehyde with 85% yield. ¹H NMR (250 MHz, CDCl₃): δ 10.00 (s, 1H), 7.58 (d, J=1.64 Hz, 2H), 7.16 (t, J=1.65 Hz, 1H), 6.89 (s, 4H), 2.26 (s, 6H), 1.96 (s, 12H). ¹³C NMR (62.5 MHz, CDCl₃): δ 192.543, 142.407, 137.436, 137.201, 137.016, 136.853, 135.660, 129.089, 128.322, 21.110, 20.837.

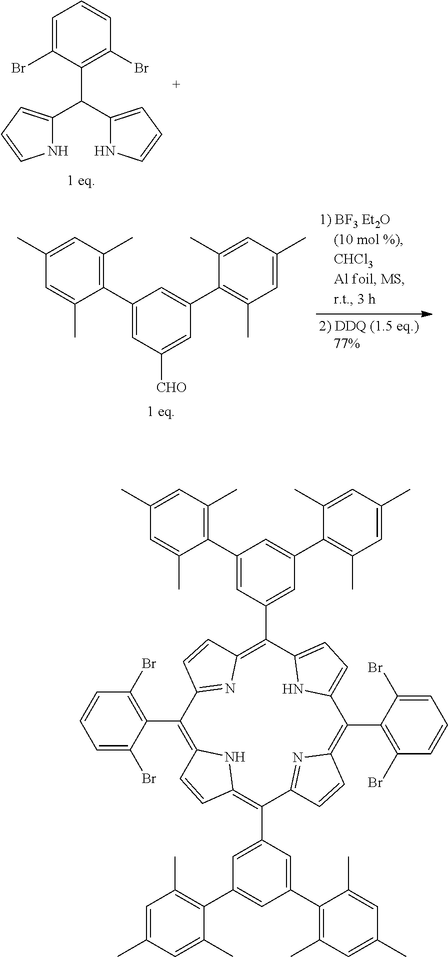

5,15-Bis(2,6-dibromophenyl)-10,20-bis(3,5-di(2,4,6-trimethylphenyl)phenyl)porphyrin were synthesized according to our previous reported procedure⁶ with 77% yield. ¹H NMR (250 MHz, CDCl₃): δ 8.93 (d, J=4.79 Hz, 4H), 8.60 (d, J=4.79 Hz, 4H), 7.92 (m, 8H), 7.42 (t, J=8 Hz, 2H), 7.27 (s, 2H), 6.90 (s, 8H), 2.31 (s, 24H), 2.20 (s, 12H), −2.65 (s, 2H). ¹³C NMR (62.5 MHz, CDCl₃): δ 143.489, 142.088, 139.913, 138.780, 136.874, 136.043, 134.291, 131.625, 131.271, 129.868, 128.610, 128.401, 120.438, 118.680, 21.342, 21.295. UV-vis (CH₂Cl₂), λₘₐₓ nm (log ε): 417 (4.23), 517 (3.87), 551 (3.34), 592 (3.38), 650 (2.92).

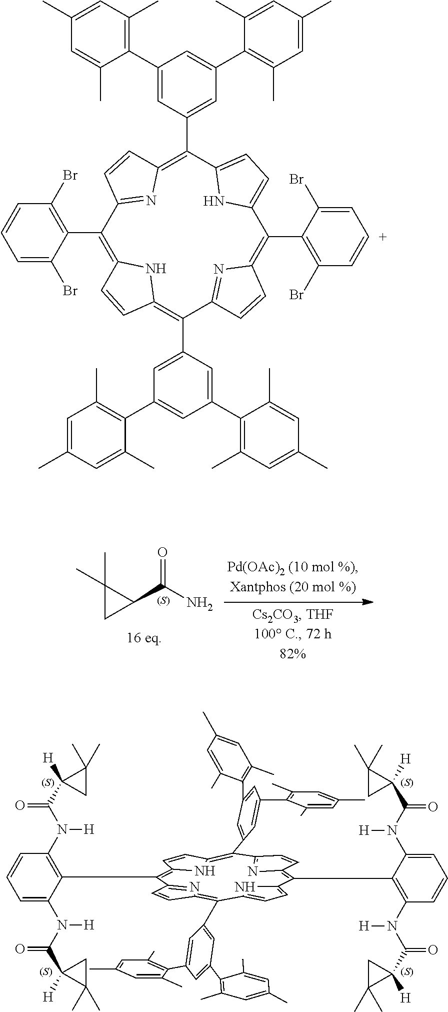

H₂P2 were synthesized according to our previous reported procedure⁶ with 82% yield. ¹H NMR (250 MHz, CDCl₃): δ 9.00 (d, J=4.75 Hz, 4H), 8.86 (d, J=4.76 Hz, 4H), 8.35 (br, 4H), 7.87 (d, J=1.26 Hz, 4H), 7.75 (t, J=8.32 Hz, 2H), 7.35 (s, 2H), 6.94 (s, 8H), 6.37 (s, 4H), 2.30 (s, 12H), 2.28 (s, 12H), 2.25 (s, 12H), 0.76 (s, 12H), 0.61 (m, 4H), −0.00 (m, 16H), −0.15 (m, 4H), −2.70 (s, 2H). ¹³C NMR (62.5 MHz, CDCl₃): δ 169.737, 141.175, 140.142, 139.341, 138.261, 137.117, 135.918, 135.856, 133.767, 130.547, 130.450, 128.378, 121.309, 117.820, 117.695, 109.063, 28.973, 26.992, 26.353, 22.540, 21.227, 21.193, 20.534, 18.277. HRMS (ESI) ([M+H]⁺) Calcd. for C104H107N8O4: 1531.8410. Found 1531.8344. UV-vis (CH₂Cl₂), λₘₐₓ nm (log ε): 424 (4.65), 516 (3.98), 552 (3.56), 592 (3.53), 645 (2.28).

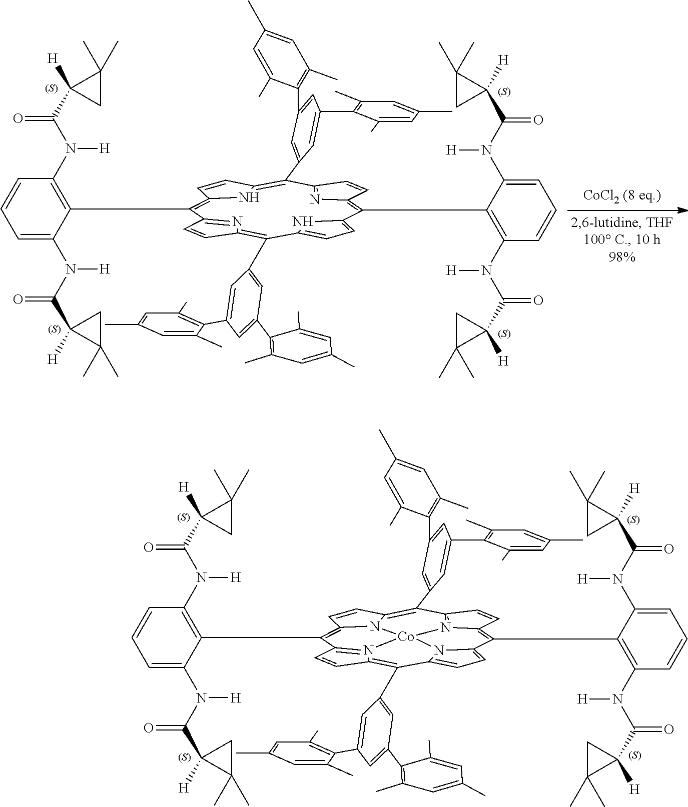

Co(P2) were synthesized according to our previous reported procedure⁶ with 98% yield. HRMS (ESI) ([M+H]⁺) Calcd. for C104H105CoN8O4: 1588.7585. Found 1588.7510. UV-vis (CH₂Cl₂), λₘₐₓ nm (log ε): 414 (4.76), 530 (4.05).

General Procedures for Cyclopropenation of Phenylacetylene Derivatives.

Catalyst (1 mol %) was placed in an oven-dried, resealable Schlenk tube. The tube was capped with a Teflon screwcap, evacuated, and backfilled with nitrogen. The screwcap was replaced with a rubber septum. 1.5 Equivalents of acetylene (0.15 mmol), 1.0 equivalents of diazo compound (0.1 mmol) in 1.0 mL α,α,α-trifluorotoluene was added once via syringe (If synthesizing larger scale, cooling while adding became necessary). Schlenk tube was capped by teflon screwcap instead of rubber septum and stirred at room temperature. After the reaction finished, the resulting mixture was concentrated and the residue was purified by flash silica gel chromatography to give the product.

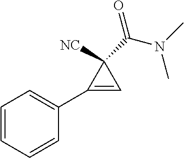 [SMILES: CN(C)C(=O)[C@]1(C#N)C=C1c1ccccc1]

### N,N-dimethyl-1-cyano-2-phenylcycloprop-2-enecarboxamide (3aa)

[α]²⁰_{D}=−18.086 (c=0.24, CHCl₃). ¹H NMR (250 MHz, CDCl₃): δ 7.65-7.61 (m, 2H), 7.46-7.35 (m, 3H), 6.89 (s, 1H), 3.36 (s, 3H), 2.92 (s, 3H). ¹³C NMR (62.5 MHz, CDCl₃): δ 166.984, 131.280, 130.382, 129.091, 123.274, 120.378, 113.326, 94.442, 38.497, 36.364, 18.996. HRMS (ESI) ([M+H]⁺) Calcd. for C13H13N2O: 213.1022. Found 213.1050. IR (neat, cm⁻¹): 1649.79, 1394.36, 1055.05, 698.730. HPLC analysis: ee=82%. Whelk-O1 (60% hexanes:40% isopropanol, 1 mL/min) tₘᵢₙₒᵣ=16.0 min, tₘₐⱼₒᵣ=23.6 min.

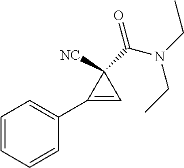 [SMILES: CCN(CC)C(=O)[C@]1(C#N)C=C1c1ccccc1]

### N,N-diethyl-1-cyano-2-phenylcycloprop-2-enecarboxamide (3ab)

[α]²⁰_{D}=−25.071 (c=0.20, CHCl₃). ¹H NMR (250 MHz, CDCl₃): δ 7.47-7.32 (m, 3H), 7.42-7.39 (m, 2H), 6.87 (s, 1H), 3.91-3.54 (m, 2H), 3.36-3.28 (m, 2H), 1.33 (t, J=6.88 Hz, 3H), 1.07 (t, J=6.96 Hz, 3H). ¹³C NMR (62.5 MHz, CDCl₃): δ 166.332, 131.259, 130.420, 129.091, 123.399, 120.725, 113.835, 94.627, 42.820, 40.629, 19.169, 13.878, 12.568. HRMS (ESI) ([M+H]⁺) Calcd. for C15H17N2O: 241.1335. Found 241.1342. IR (neat, cm⁻¹): 1634.55, 1428.37, 1275.89, 729.77, 697.58. HPLC analysis: ee=80%. Whelk-O1 (60% hexanes:40% isopropanol, 1 mL/min) tₘᵢₙₒᵣ=12.2 min, tₘₐⱼₒᵣ=18.6 min.

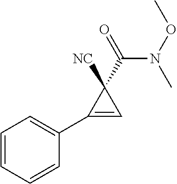 [SMILES: CON(C)C(=O)[C@]1(C#N)C=C1c1ccccc1]

### N-methoxy-N-methyl-1-cyano-2-phenylcycloprop-2-enecarboxamide (3ac)

[α]²⁰_{D}=+34.076 (c=0.20, CHCl₃). ¹H NMR (250 MHz, CDCl₃): δ 7.71-7.54 (m, 2H), 7.42-7.40 (m, 3H), 6.92 (s, 1H), 3.91 (s, 3H), 3.17 (s, 3H). ¹³C NMR (62.5 MHz, CDCl₃): δ 168.835, 131.377, 130.436, 129.156, 122.928, 111.925, 94.169, 61.666, 33.344, 18.633. HRMS (ESI) ([M+H]⁺) Calcd. for C13H13N2O2: 229.0972. Found 2229.0971. IR (neat, cm⁻¹): 1669.65, 1266.01, 736.66, 702.19. HPLC analysis: ee=83%. Whelk-O1 (60% hexanes:40% isopropanol, 1 mL/min) tₘᵢₙₒᵣ=11.0 min, tₘₐⱼₒᵣ=19.7 min.

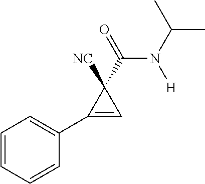 [SMILES: CC(C)NC(=O)[C@]1(C#N)C=C1c1ccccc1]

### N-isopropyl-1-cyano-2-phenylcycloprop-2-enecarboxamide (3ad)

[α]²⁰_{D}=+79.579 (c=0.5, CHCl₃). ¹H NMR (250 MHz, CDCl₃): δ 6.11-5.86 (m, 1H), 7.59-7.47 (m, 2H), 7.47-7.34 (m, 3H), 6.85 (s, 1H), 4.14-4.00 (m, 1H), 1.15 (d, J=6.31 Hz, 3H), 1.12 (d, J=6.31 Hz, 3H). ¹³C NMR (62.5 MHz, CDCl₃): δ 165.982, 131.630, 130.405, 129.270, 122.313, 120.157, 111.377, 93.545, 42.783, 22.771, 22.691, 20.400. HRMS (ESI) ([M+H]⁺) Calcd. for C14H15N2O: 227.1179. Found 227.1179. IR (neat, cm⁻¹): 1668.00, 1518.12, 1265.99, 737.43. HPLC analysis: ee=96%. Whelk-O1 (60% hexanes:40% isopropanol, 1 mL/min) tₘᵢₙₒᵣ=9.2 min, tₘₐⱼₒᵣ=20.5 min.

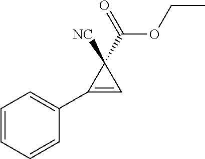 [SMILES: CCOC(=O)[C@]1(C#N)C=C1c1ccccc1]

### Ethyl 1-cyano-2-phenylcycloprop-2-enecarboxylate (3ae)

[α]²⁰_{D}=+68.324 (c=0.5, CHCl₃). ¹H NMR (250 MHz, CDCl₃): δ 7.61-7.49 (m, 2H), 7.49-7.37 (m, 3H), 6.86 (s, 1H), 4.22 (q, J=7.13 Hz, 2H), 1.26 (t, J=7.13 Hz, 3H). ¹³C NMR (62.5 MHz, CDCl₃): δ 168.924, 131.889, 130.593, 129.455, 122.184, 119.371, 111.318, 93.562, 62.928, 19.477, 14.381. HRMS (ESI) ([M+H]⁺) Calcd. for C13H12NO2: 214.0863. Found 213.0864. IR (neat, cm⁻¹): 1742.06, 1256.56, 1196.37, 1079.29, 701.10. HPLC analysis: ee=93%. Whelk-O1 (60% hexanes:40% isopropanol, 1 mL/min) tₘᵢₙₒᵣ=7.3 min, tₘₐⱼₒᵣ=10.8 min.

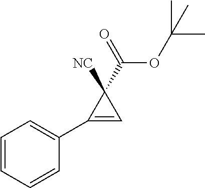 [SMILES: CC(C)(C)OC(=O)[C@]1(C#N)C=C1c1ccccc1]

### (R)-tert-butyl 1-cyano-2-phenylcycloprop-2-enecarboxylate (3af)

[α]²⁰_{D}=+44.009 (c=0.2, CHCl₃). ¹H NMR (250 MHz, CDCl₃): δ 7.48-7.39 (m, 3H), 7.59-7.48 (m, 2H), 6.83 (s, 1H), 1.45 (s, 9H). ¹³C NMR (62.5 MHz, CDCl₃): δ 167.755, 131.612, 130.373, 129.309, 122.268, 119.616, 111.342, 93.662, 83.708, 28.000, 20.096. HRMS (ESI) ([M+H]⁺) Calcd. for C15H16NO2: 242.1176. Found 242.1184. IR (neat, cm⁻¹): 1723.00, 1281.48, 1158.67, 701.78. HPLC analysis: ee=98%. Whelk-O1 (60% hexanes:40% isopropanol, 1 mL/min) tₘᵢₙₒᵣ=5.9 min, tₘₐⱼₒᵣ=7.2 min.

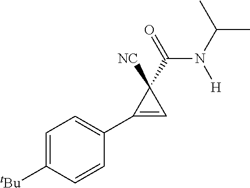 [SMILES: CC(C)NC(=O)[C@]1(C#N)C=C1c1ccc(C(C)(C)C)cc1]

### N-isopropyl-1-cyano-2-(4-tert-butylphenyl)cycloprop-2-enecarboxamide (3bd)

[α]²⁰_{D}=+56.267 (c=0.2, CHCl₃). ¹H NMR (250 MHz, CDCl₃): δ 7.52-7.37 (m, 4H), 6.78 (s, 1H), 5.95 (d, J=7.54 Hz, 1H), 4.05 (m, 1H), 1.26 (s, 9H), 1.13 (d, J=6.16 Hz, 3H), 1.11 (d, J=6.16 Hz, 3H). ¹³C NMR (62.5 MHz, CDCl₃): δ 166.242, 155.378, 130.251, 126.320, 120.261, 119.382, 111.282, 92.439, 42.714, 35.189, 31.141, 22.760, 22.681, 20.330. HRMS (ESI) ([M+H]⁺) Calcd. for C18H23N2O: 283.1805. Found 283.1805. IR (neat, cm⁻¹): 1669.72, 1518.49, 1265.17, 734.80, 704.08. HPLC analysis: ee=90%. Whelk-O1 (60% hexanes:40% isopropanol, 1 mL/min) tₘᵢₙₒᵣ=8.1 min, tₘₐⱼₒᵣ=18.5 min.

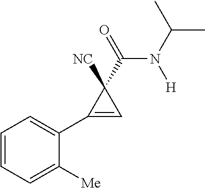 [SMILES: Cc1ccccc1C1=C[C@@]1(C#N)C(=O)NC(C)C]

### N-isopropyl-1-cyano-2-o-tolylcycloprop-2-enecarboxamide (3cd)

[α]²⁰_{D}=+29.138 (c=0.2, CHCl₃). ¹H NMR (250 MHz, CDCl₃): δ 7.39-7.21 (m, 4H), 6.88 (s, 1H), 6.19-5.88 (m, 1H), 4.13-4.00 (m, 1H), 2.44 (s, 3H), 1.14 (d, J=5.75 Hz, 3H), 1.11 (d, J=5.75 Hz, 3H). ¹³C NMR (62.5 MHz, CDCl₃): δ 166.133, 140.595, 131.593, 130.940, 130.661, 126.547, 121.379, 120.317, 110.509, 94.828, 42.780, 22.746, 22.655, 20.019. IR (neat, cm⁻¹): 1675.91, 1518.48, 1265.02, 734.06, 704.29. HRMS (ESI) ([M+H]⁺) Calcd. for C15H17N2O: 241.1335. Found 241.1335. HPLC analysis: ee=92%. Whelk-O1 (60% hexanes:40% isopropanol, 1 mL/min) tₘᵢₙₒᵣ=9.2 min, tₘₐⱼₒᵣ=29.1 min.

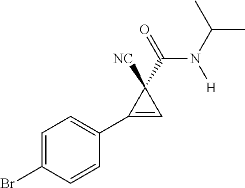 [SMILES: CC(C)NC(=O)[C@]1(C#N)C=C1c1ccc(Br)cc1]

### N-isopropyl-1-cyano-2-(4-bromophenyl)cycloprop-2-enecarboxamide (3dd)

[α]²⁰_{D}=+38.181 (c=0.2, CHCl₃). ¹H NMR (250 MHz, CDCl₃): δ 6.09-5.90 (m, 1H), 7.61-7.50 (m, 2H), 6.89 (s, 1H), 7.37 (d, J=8.39 Hz, 2H), 4.12-4.00 (m, 1H), 1.14 (d, J=6.00 Hz, 3H), 1.12 (d, J=6.00 Hz, 3H). ¹³C NMR (62.5 MHz, CDCl₃): δ 165.556, 132.652, 131.636, 126.391, 121.316, 119.869, 110.513, 94.443, 42.880, 22.745, 22.673. HRMS (ESI) ([M+H]⁺) Calcd. for C14H14BrN2O: 305.0284. Found 305.0277. IR (neat, cm⁻¹): 1683.59, 1519.98, 1264.96, 734.45, 704.09. HPLC analysis: ee=97%. Whelk-O1 (60% hexanes:40% isopropanol, 1 mL/min) tₘᵢₙₒᵣ=8.5 min, tₘₐⱼₒᵣ=19.9 min.

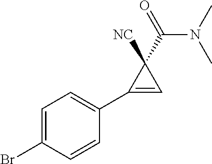 [SMILES: CN(C)C(=O)[C@]1(C#N)C=C1c1ccc(Br)cc1]

### N,N-dimethyl-1-cyano-2-(4-bromophenyl)cycloprop-2-enecarboxamide (3da)

[α]²⁰_{D}=−75.358 (c=0.20, CHCl₃). ¹H NMR (250 MHz, CDCl₃): δ 7.60-7.46 (m, 4H), 6.90 (s, 1H), 3.35 (s, 3H), 2.92 (d, 3H). ¹³C NMR (62.5 MHz, CDCl₃): δ 166.705, 132.439, 131.798, 125.995, 122.244, 119.994, 112.954, 95.117, 38.429, 36.306, 19.076. HRMS (ESI) ([M+H]⁺) Calcd. for C13H12BrN2O: 291.0128. Found 291.0133. IR (neat, cm⁻¹): 1645.03, 1395.82, 1069.39, 1010.81, 827.70. HPLC analysis: ee=83%. Whelk-O1 (60% hexanes:40% isopropanol, 1 mL/min) tₘᵢₙₒᵣ=16.3 min, tₘₐⱼₒᵣ=20.9 min.

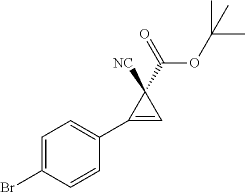 [SMILES: CC(C)(C)OC(=O)[C@]1(C#N)C=C1c1ccc(Br)cc1]

### (R)-tert-butyl 2-(4-bromophenyl)-1-cyanocycloprop-2-enecarboxylate (3df)

[α]²⁰_{D}=+58.66° (c=0.25, CHCl₃). ¹H NMR (250 MHz, CDCl₃): δ 7.58 (d, J=8.5 Hz, 2H), 7.38 (d, J=8.5 Hz, 2H), 6.89 (s, 1H), 1.43 (s, 9H). ¹³C NMR (62.5 MHz, CDCl₃): δ 167.376, 132.697, 131.570, 126.388, 121.255, 119.235, 110.623, 94.648, 83.912, 27.964, 20.126. HRMS (ESI) ([M+Na]⁺) Calcd. for C15H14BrNO2Na: 342.0100. Found 342.0106. IR (neat, cm⁻¹):1718.50, 1291.82, 1162.74, 834.15, 722.63, 691.63. HPLC analysis: ee=99%. Whelk-O1 (60% hexanes: 40% isopropanol, 1 mL/min) tₘᵢₙₒᵣ=6.0 min, tₘₐⱼₒᵣ=7.4 min.

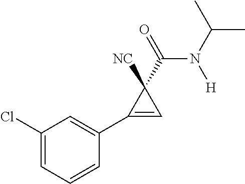 [SMILES: CC(C)NC(=O)[C@]1(C#N)C=C1c1cccc(Cl)c1]

### N-isopropyl-1-cyano-2-(3-chlorophenyl)cycloprop-2-enecarboxamide (3ed)

[α]²⁰_{D}=+20.804 (c=0.2, CHCl₃). ¹H NMR (250 MHz, CDCl₃): δ 7.49 (s, 1H), 7.46-7.27 (m, 3H), 6.92 (s, 1H), 6.16-5.92 (m, 1H), 4.13-4.02 (m, 1H), 1.16 (d, J=6.59 Hz, 3H), 1.13 (d, J=6.59 Hz, 3H). ¹³C NMR (62.5 MHz, CDCl₃): δ 164.389, 134.237, 130.644, 129.509, 129.029, 127.340, 123.048, 118.758, 109.223, 94.204, 41.881, 21.681, 21.623, 19.425. HRMS (ESI) ([M+H]⁺) Calcd. for C14H14ClN2O: 261.0789. Found 261.0789. IR (neat, cm⁻¹): 1656.85, 1531.45, 1196.51, 800.70. HPLC analysis: ee=98%. Whelk-O1 (60% hexanes:40% isopropanol, 1 mL/min) tₘᵢₙₒᵣ=8.4 min, tₘₐⱼₒᵣ=19.9 min.

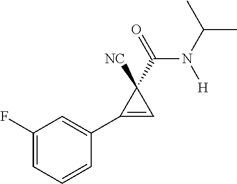 [SMILES: CC(C)NC(=O)[C@]1(C#N)C=C1c1cccc(F)c1]

### N-isopropyl-1-cyano-2-(3-fluorophenyl)cycloprop-2-enecarboxamide (3fd)

[α]²⁰_{D}=+49.229 (c=0.2, CHCl₃). ¹H NMR (250 MHz, CDCl₃): δ 7.46-7.34 (m, 1H), 7.30 (dd, J=7.63, 1.04 Hz, 1H), 7.26-7.06 (m, 2H), 6.92 (s, 1H), 6.17-5.91 (m, 1H), 4.06 (d, J=13.29 Hz, 1H), 1.16 (d, J=6.00 Hz, 3H), 1.13 (d, J=6.00 Hz, 3H). ¹³C NMR (62.5 MHz, CDCl₃): δ 165.486, 162.810 (d, J=247 Hz), 160.831, 131.039 (d, J=8.3 Hz), 126.150 (d, J=3.1 Hz), 124.327 (d, J=8.6 Hz), 119.828, 118.801 (d, J=21 Hz), 117.007 (d, J=22.9 Hz), 110.423 (d, J=3.3 Hz), 95.134, 42.912, 22.723, 22.656, 20.541. HRMS (ESI) ([M+H]⁺) Calcd. for C14H14FN2O: 245.1085. Found 245.1079. IR (neat, cm⁻¹): 1652.80, 1531.17, 1248.15, 851.04. HPLC analysis: ee=98%. Whelk-O1 (60% hexanes:40% isopropanol, 1 mL/min) tₘᵢₙₒᵣ=7.9 min, tₘₐⱼₒᵣ=15.9 min.

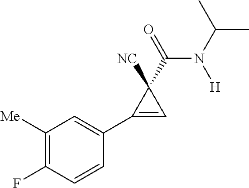 [SMILES: Cc1cc(C2=C[C@@]2(C#N)C(=O)NC(C)C)ccc1F]

### N-isopropyl-1-cyano-2-(4-fluoro-3-methylphenyl)cycloprop-2-enecarboxamide (3gd)

[α]²⁰_{D}=+54.258 (c=0.2, CHCl₃). ¹H NMR (250 MHz, CDCl₃): δ 7.14-6.97 (m, 1H), 7.48-7.25 (m, 2H), 6.79 (s, 1H), 5.98 (d, J=7.13 Hz, 1H), 4.12-3.98 (m, 1H), 2.24 (s, 3H), 1.13 (d, J=6.85 Hz, 3H), 1.12 (d, J=6.85 Hz, 3H). ¹³C NMR (62.5 MHz, CDCl₃): δ 165.942, 161.157, 133.674 (d, J=6.2 Hz), 129.918 (d, J=8.9 Hz), 126.570 (d, J=18 Hz), 120.096, 118.263, 118.206, 116.487, 116.111, 110.641, 92.809, 42.784, 22.731, 22.664, 20.475, 14.527 (d, J=3.5 Hz). HRMS (ESI) ([M+H]⁺) Calcd. for C15H16FN2O: 259.1241. Found 259.1245. IR (neat, cm⁻¹): 1670.08, 1496.23, 1265.01, 733.80. HPLC analysis: ee=94%. Whelk-O1 (60% hexanes:40% isopropanol, 1 mL/min) tₘᵢₙₒᵣ=8.5 min, tₘₐⱼₒᵣ=19.6 min.

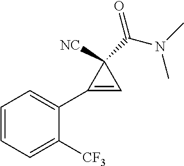 [SMILES: CN(C)C(=O)[C@]1(C#N)C=C1c1ccccc1C(F)(F)F]

### N,N-dimethyl-1-cyano-2-(2-trifluoromethylphenyl)cycloprop-2-enecarboxamide (3ha)

[α]²⁰_{D}=−96.123 (c=0.2, CHCl₃). ¹H NMR (250 MHz, CDCl₃): δ7.90 (d, J=7.55 Hz, 1H), 7.73 (d, J=7.66 Hz, 1H), 7.63 (t, J=7.49 Hz, 1H), 7.54 (t, J=7.49 Hz, 1H), 7.06 (s, 1H), 3.34 (s, 3H), 2.92 (s, 3H). ¹³C NMR (62.5 MHz, CDCl₃): δ 166.435, 133.102, 132.545, 131.242, 130.061 (q, J=32 Hz), 126.684 (q, J=5.5 Hz), 121.065, 119.712, 111.048, 99.681 (q, J=4.2 Hz), 38.307, 36.232, 19.299. HRMS (ESI) ([M+H]⁺) Calcd. for C14H12F3N2O: 281.0896. Found 281.0895. IR (neat, cm⁻¹): 1651.06, 1314.48, 1171.49, 1128.54, 1059.92, 768.48. HPLC analysis: ee=91%. Whelk-O1 (60% hexanes:40% isopropanol, 1 mL/min) tₘᵢₙₒᵣ=11.2 min, tₘₐⱼₒᵣ=13.1 min.

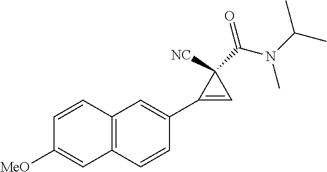 [SMILES: COc1ccc2cc(C3=C[C@@]3(C#N)C(=O)N(C)C(C)C)ccc2c1]

### N-isopropyl-1-cyano-2-(6-methoxynaphthalen-2-yl)cycloprop-2-enecarboxamide (31d)

[α]²⁰_{D}=+23.11° (c=0.4, CHCl₃). ¹H NMR (250 MHz, CDCl₃): δ 7.88 (s, 1H), 7.72 (d, J=8.22 Hz, 2H), 7.54 (d, J=8.50 Hz, 1H), 7.18-7.02 (m, 2H), 6.86 (s, 1H), 5.96 (d, J=7.77 Hz, 1H), 4.14-4.01 (m, 1H), 3.88 (s, 3H), 1.14 (d, J=6.54 Hz, 3H), 1.11 (d, J=6.54 Hz, 3H). ¹³C NMR (62.5 MHz, CDCl₃): δ 166.330, 159.554, 136.139, 131.133, 130.309, 128.339, 128.019, 126.750, 120.323, 120.138, 117.207, 111.800, 105.999, 92.616, 55.496, 42.723, 22.783, 22.703, 20.623. HRMS (ESI) ([M+H]⁺) Calcd. for C19H19N2O2: 307.1441. Found 307.1440. IR (neat, cm⁻¹): 1653.00, 1519.69, 1265.62, 733.68. HPLC analysis: ee=88%. ODH (95% hexanes:5% isopropanol, 1 mL/min) tₘᵢₙₒᵣ=45.5 min, tₘₐⱼₒᵣ=57.3 min.

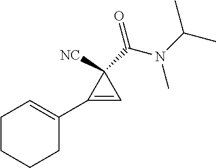 [SMILES: CC(C)N(C)C(=O)[C@]1(C#N)C=C1C1=CCCCC1]

### N-isopropyl-1-cyano-2-cyclohexenylcycloprop-2-enecarboxamide (3jd)

[α]²⁰_{D}=+41.53° (c=0.5, CHCl₃). ¹H NMR (250 MHz, CDCl₃): δ 6.48 (s, 1H), 6.19 (s, 1H), 6.00-5.79 (m, 1H), 4.12-3.98 (m, 1H), 2.41-2.07 (m, 4H), 1.63 (m, 6H), 1.12 (d, J=6.55 Hz, 6H). ¹³C NMR (62.5 MHz, CDCl₃): δ 166.253, 140.367, 122.133, 112.235, 91.188, 42.620, 26.553, 25.915, 22.742, 22.680, 21.893, 21.391, 20.043, 14.251. IR (neat, cm⁻¹): 1662.18, 1525.05, 1266.05, 738.20. HRMS (ESI) ([M+H]⁺) Calcd. for C14H19N2O: 231.1492. Found 231.1501. HPLC analysis: ee=89%. Whelk-O1 (60% hexanes:40% isopropanol, 1 mL/min) tₘᵢₙₒᵣ=7.6 min, tₘₐⱼₒᵣ=10.8 min.

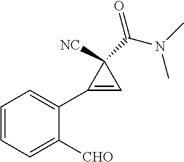 [SMILES: CN(C)C(=O)[C@]1(C#N)C=C1c1ccccc1C=O]

### N,N-dimethyl-1-cyano-2-(2-formylphenyl)cycloprop-2-enecarboxamide (3ka)

[α]²⁰_{D}=−35.678 (c=0.20, CHCl₃). ¹H NMR (250 MHz, CDCl₃): δ 10.25 (s, 1H), 7.98-7.80 (m, 2H), 7.66 (dtd, J=17.48, 7.32, 1.05 Hz, 2H), 7.21 (s, 1H), 3.35 (s, 3H), 2.93 (s, 3H). ¹³C NMR (62.5 MHz, CDCl₃): δ 190.758, 166.640, 135.137, 134.404, 133.194, 132.836, 131.528, 123.119, 119.909, 111.190, 100.463, 38.367, 36.272, 19.345. HRMS (ESI) ([M+H]⁺) Calcd. for C14H13N2O2: 241.0972. Found 241.0981. IR (neat, cm⁻¹): 1697.55, 1646.74, 1395.61, 1199.19, 732.55. HPLC analysis: ee=82%. ADH (80% hexanes:20% isopropanol, 1 mL/min) tₘᵢₙₒᵣ=12.6 min, tₘₐⱼₒᵣ=17.9 min.

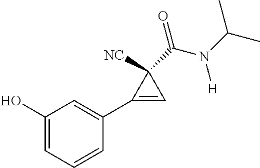 [SMILES: CC(C)NC(=O)[C@]1(C#N)C=C1c1cccc(O)c1]

### N-isopropyl-1-cyano-2-(3-hydroxyphenyl)cycloprop-2-enecarboxamide (31d)

[α]²⁰_{D}=+74.343 (c=0.2, CHCl₃). ¹H NMR (250 MHz, CD₃COCD₃): δ 7.38 (s, 1H), 7.28 (t, J=7.84 Hz, 1H), 7.08-6.83 (m, 3H), 6.72-6.70 (m, 1H), 4.02-3.74 (m, 1H), 3.00-2.62 (m, 1H), 0.98 (d, J=6.61 Hz, 3H), 0.91 (d, J=6.61 Hz, 3H). ¹³C NMR (62.5 MHz, CD3COCD3): δ 167.561, 158.920, 131.528, 124.934, 122.357, 119.622, 117.339, 113.344, 96.703, 42.774, 22.550. HRMS (ESI) ([M+H]⁺) Calcd. for C14H15N2O2: 243.1128. Found 243.1123. IR (neat, cm⁻¹): 3371.30, 1653.10, 1539.11, 1265.92, 736.78. HPLC analysis: ee=92%. Whelk-O1 (60% hexanes:40% isopropanol, 1 mL/min) tₘᵢₙₒᵣ=6.8 min, tₘₐⱼₒᵣ=14.3 min.

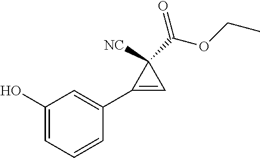 [SMILES: CCOC(=O)[C@]1(C#N)C=C1c1cccc(O)c1]

### ethyl 1-cyano-2-(3-hydroxyphenyl)cycloprop-2-enecarboxylate (31e)

[α]²⁰_{D}=+56.775 (c=0.2, CHCl₃). ¹H NMR (250 MHz, CDCl₃): δ 7.27 (t, J=7.87 Hz, 1H), 7.09 (d, J=7.65 Hz, 1H), 6.99-6.85 (m, 2H), 6.84 (s, 1H), 5.77 (s, 1H), 4.22 (q, J=7.13 Hz, 2H), 1.25 (t, J=7.13 Hz, 3H). ¹³C NMR (62.5 MHz, CDCl₃): δ 169.089, 156.446, 130.641, 123.002, 122.823, 119.239, 119.156, 116.865, 110.826, 100.043, 93.506, 63.075, 19.462, 14.242. HRMS (ESI) ([M+H]⁺) Calcd. for C13H12NO3: 230.0812. Found 230.0821. IR (neat, cm⁻¹): 3410.39, 1725.09, 1446.42, 1271.80, 1078.56, 855.39. HPLC analysis: ee=88%. Whelk-O1 (60% hexanes:40% isopropanol, 1 mL/min) tₘᵢₙₒᵣ=5.8 min, tₘₐⱼₒᵣ=8.8 min.

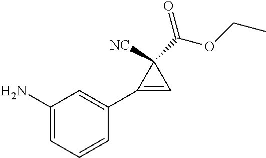 [SMILES: CCOC(=O)[C@]1(C#N)C=C1c1cccc(N)c1]

### ethyl 1-cyano-2-(3-aminophenyl)cycloprop-2-enecarboxylate (3me)

[α]²⁰_{D}=+23.052 (c=0.2, CHCl₃). ¹H NMR (250 MHz, CDCl₃): δ 7.25-7.13 (m, 1H), 6.95 (d, J=7.61 Hz, 1H), 6.85-6.67 (m, 2H), 6.79 (s, 1H), 4.21 (q, J=7.13 Hz, 2H), 3.48 (br, 2H), 1.25 (t, J=7.13 Hz, 3H). ¹³C NMR (62.5 MHz, CDCl₃): δ 168.906, 130.241, 122.743, 120.776, 119.389, 118.428, 116.175, 111.263, 92.994, 62.764, 19.360, 14.271. IR (neat, cm⁻¹): 1731.90, 1265.15, 735.57, 703.37. HRMS (ESI) ([M+H]⁺) Calcd. for C13H13N2O2: 229.0972. Found 229.0967. HPLC analysis: ee=95%. Whelk-O1 (60% hexanes:40% isopropanol, 1 mL/min) tₘᵢₙₒᵣ=10.6 min, tₘₐⱼₒᵣ=21.2 min.

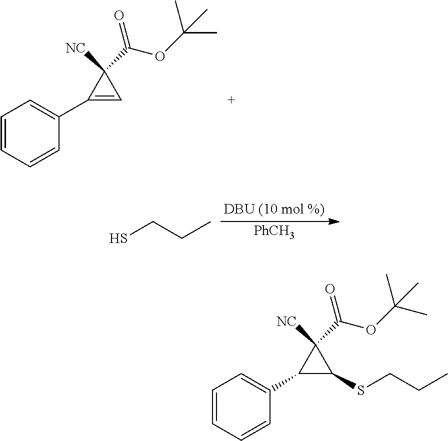

General Procedure for Alkylthiocyclopropane Synthesis:

To a solution of tert-butyl 1-cyano-2-phenylcycloprop-2-enecarboxylate (36 mg, 0.15 mmol) in 7.5 mL of toluene at −30° C. was added DBU (2.3 μL, 0.015 mmol) and 1-propanethiol (20 μL, 0.225 mmol). The temperature was raised to 0° C. and the mixture was stirred overnight and concentrated to remove toluene, followed by purification of the residue by column chromatography 46.5 mg (98%) of tert-butyl 1-cyano-2-phenyl-3-(propylthio)cyclopropanecarboxylate was isolated as solo diastereomer.

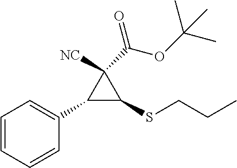 [SMILES: CCCS[C@H]1[C@H](c2ccccc2)[C@]1(C#N)C(=O)OC(C)(C)C]

### (1S,2R,3S)-tert-butyl 1-cyano-2-phenyl-3-(propylthio)cyclopropanecarboxylate (4a)

[α]²⁰_{D}=−70.334 (c=0.25, CHCl₃). ¹H NMR (250 MHz, CDCl₃): δ 7.23 (m, 5H), 3.44 (d, J=7.94 Hz, 1H), 3.16 (d, J=7.96 Hz, 1H), 2.70 (t, J=7.28 Hz, 2H), 1.67 (m, 2H), 1.13 (s, 9H), 1.00 (t, J=7.25 Hz, 1H). ¹³C NMR (62.5 MHz, CDCl₃): δ 162.258, 131.725, 129.034, 128.469, 116.848, 84.190, 42.797, 34.975, 32.559, 31.975, 27.616, 22.798, 13.553. IR (neat, cm⁻¹): 1731.64, 1317.49, 1143.06, 735.63. (1S,2R,3S) stereoisomer was determined by NOE analysis and the chirality from (R)-tert-butyl 1-cyano-2-phenylcycloprop-2-enecarboxylate. HRMS (ESI) ([M+H]⁺) Calcd. for C18H24NO2S: 318.1522. Found 318.1522. HPLC analysis: ee=98%. Whelk-O1 (99% hexanes:1% isopropanol, 1 mL/min) tₘₐⱼₒᵣ=26.0 min, tₘᵢₙₒᵣ=29.9 min.

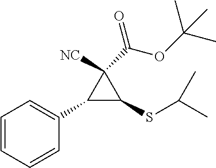 [SMILES: CC(C)S[C@H]1[C@H](c2ccccc2)[C@]1(C#N)C(=O)OC(C)(C)C]

### tert-butyl 1-cyano-2-(isopropylthio)-3-phenylcyclopropanecarboxylate (4b)

[α]²⁰_{D}=−26.794 (c=0.25, CHCl₃). ¹H NMR (250 MHz, CDCl₃): δ 7.23-7.20 (m, 5H), 3.46 (d, J=8.04 Hz, 1H), 3.15 (d, J=8.04 Hz, 1H), 3.29-3.04 (m, 1H), 1.34 (d, J=6.66 Hz, 6H), 1.14 (s, 9H). ¹³C NMR (62.5 MHz, CDCl₃): δ 162.258, 131.725, 129.034, 128.469, 116.848, 84.190, 42.797, 34.975, 32.559, 31.975, 27.616, 22.798, 13.553. HRMS (ESI) ([M+H]⁺) Calcd. for C18H24NO2S: 318.1522. Found 318.1523. IR (neat, cm⁻¹): 1730.03, 1265.34, 1142.31, 736.97. HPLC analysis: ee=98%. Whelk-O1 (99% hexanes:1% isopropanol, 1 mL/min) tₘₐⱼₒᵣ=23.4 min, tₘᵢₙₒᵣ=30.5 min.

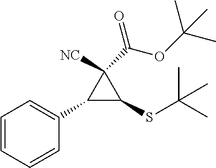 [SMILES: CC(C)(C)OC(=O)[C@]1(C#N)[C@@H](SC(C)(C)C)[C@@H]1c1ccccc1]

### tert-butyl 2-(tert-butylthio)-1-cyano-3-phenylcyclopropanecarboxylate (4c)

Reaction was carried out in the presence of 1 eq. of DBU for 48 hours. [α]²⁰_{D}=−14.067 (c=0.25, CHCl₃). ¹H NMR (250 MHz, CDCl₃): δ 7.24-7.22 (m, 5H), 3.43 (d, J=8.24 Hz, 1H), 3.09 (d, J=8.23 Hz, 1H), 1.41 (s, 9H), 1.15 (s, 9H). ¹³C NMR (62.5 MHz, CDCl₃): δ 162.365, 131.820, 129.048, 128.470, 128.378, 116.994, 84.147, 44.712, 41.078, 32.394, 30.994, 30.821, 27.646. HRMS (ESI) ([M+H]⁺) Calcd. for C19H26NO2S: 332.1679. Found 332.1671. IR (neat, cm⁻¹): 1728.17, 1369.08, 1315.50, 1140.73, 696.10. HPLC analysis: ee=98%. Whelk-O1 (99% hexanes:1% isopropanol, 1 mL/min) tₘₐⱼₒᵣ=20.8 min, tₘᵢₙₒᵣ=27.9 min.

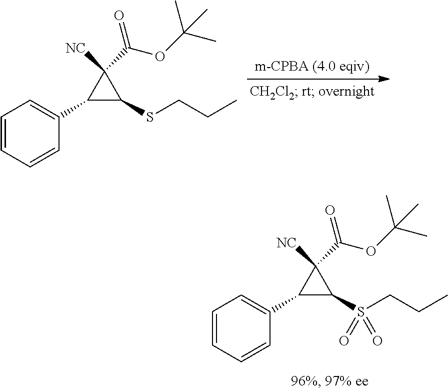 [SMILES: CCCS[C@H]1[C@H](c2ccccc2)[C@]1(C#N)C(=O)OC(C)(C)C ; CCCS(=O)(=O)[C@H]1[C@H](c2ccccc2)[C@]1(C#N)C(=O)OC(C)(C)C]

### (1S,2R,3S)-tert-butyl 1-cyano-2-phenyl-3(propylsulfonyl)cyclopropanecarboxylate (5a)

tert-butyl 1-cyano-2-phenyl-3-(propylthio)cyclopropanecarboxylate (35 mg, 0.11 mmol) was oxidized by 4 eq. of m-CPBA in 5 mL of DCM overnight. After wash with saturated Na₂SO₃ and Na₂CO₃ solution, column chromatography afforded 37 mg (96%) of tert-butyl 1-cyano-2-phenyl-3-(propylsulfonyl)cyclopropanecarboxylate as a white solid. enantio excess ratio was remained unchanged. [α]²⁰_{D}=−13.397 (c=0.2, CHCl₃). ¹H NMR (250 MHz, CDCl₃): δ 7.29-7.20 (m, 5H), 3.89 (d, J=7.94 Hz, 1H), 3.84 (d, J=7.79 Hz, 1H), 2.08-1.83 (m, 2H), 3.28-3.10 (m, 2H), 1.14 (s, 9H), 1.09 (t, J=7.74 Hz, 3H). ¹³C NMR (62.5 MHz, CDCl₃): δ 159.902, 129.168, 128.980, 128.747, 114.039, 85.963, 55.758, 44.329, 36.593, 27.501, 15.771, 13.213. HRMS (ESI) ([M+Na]⁺) Calcd. for C18H23NO4SNa: 372.1240. Found 372.1238. IR (neat, cm⁻¹): 1732.34, 1316.56, 1249.16, 1138.88. HPLC analysis: ee=97%. Whelk-O1 (90% hexanes:10% isopropanol, 1 mL/min) t=40.6 min, tₘᵢₙₒᵣ=51.7 min.
a) Cavender, C. J.; Shiner, V. J., Jr. J. Org. Chem. 1972, 37, 3567. (b) Fritschi, S.; Vasella, A. Helv. Chim. Acta 1991, 74, 2024. (c) Charette, A. B.; Wurz, R. P.; Ollevier, T. J. Org. Chem. 2000, 65, 9252.
b) Wurz, R. P.; Lin, W.; Charette, A. B. Tetrahedron Lett. 2003, 44, 8845.
c) Marcoux, D.; Azzi, S.; Charette, A. B. J. Am. Soc. Chem. 2009, 131, 6970.
d) Basheer, A.; Yamataka, H.; Ammal, S.C.; Rappoport, Z. J. Org. Chem. 2007, 72, 5297.
e) Sawamura, M.; Nakayama, Y.; Kato, T.; Ito, Y. J. Org. Chem. 1995, 60, 1727.
f) Chen, Y.; Fields, K. B.; Zhang, X. P. J. Am. Soc. Chem. 2004, 126, 14718.

### X-ray Crystallography

The X-ray diffraction data were collected using Bruker-AXS SMART-APEXII CCD diffractometer (CuKα, λ=1.54178 Å). Indexing was performed using APEX2 [1] (Difference Vectors method). Data integration and reduction were performed using SaintPlus 6.01 [2]. Absorption correction was performed by multi-scan method implemented in SADABS [3]. Space groups were determined using XPREP implemented in APEX2 [1]. The structure was solved using SHELXS-97 (direct methods) and refined using SHELXL-97 (full-matrix least-squares on F²) contained in APEX2 [1] and WinGX v 1.70.01 [4,5,6,7] programs packages. All non-hydrogen atoms were refined anisotropically. Absolute configuration for Br-derivative was established by anomalous-dispersion effects in diffraction measurements on the single crystal. The absolute configuration for the parent compound (containing only light atoms C, H, N, O) was confirmed through the absolute configuration of Br-derivative.

For the structure of S-derivative, atoms C6a, C6b, C10a and C10b of disordered phenyl and ester groups were refined isotropically. Both groups are disordered over two positions with 50/50 occupancies. Crystal data and refinement conditions are shown in Tables 1, 2 and 3. Absolute configuration was established by anomalous-dispersion effects.
[1] Bruker (2010). APEX2). Bruker AXS Inc., Madison, Wis., USA.
[2] Bruker (2009). SAINT. Data Reduction Software. Bruker AXS Inc., Madison, Wis., USA.
[3] Sheldrick, G. M. (2008). SADABS. Program for Empirical Absorption Correction. University of Gottingen, Germany.
[4] Farrugia L. J. Appl. Cryst. (1999). 32, 837±838
[5] Sheldrick, G. M. (1997) SHELXL-97. Program for the Refinement of Crystal
[6] Sheldrick, G. M. (1990) Acta Cryst. A46, 467-473
[7] Sheldrick, G. M. (2008). Acta Cryst. A64, 112-122.

TABLE 1

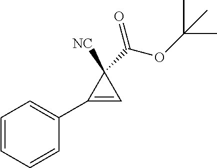 [SMILES: CC(C)(C)OC(=O)[C@]1(C#N)C=C1c1ccccc1]

Crystal data and structure refinement for compound 3af

| | |
|---|---|
| Identification code | 3af |
| Empirical formula | C15 H15 N O2 |
| Formula weight | 241.28 |
| Temperature | 100(2) K |
| Wavelength | 1.54178 A |
| Crystal system, space group | Orthorhombic, P2₁2₁2₁ |
| Unit cell dimensions | a = 5.4994(1) A alpha = 90 deg. |
| | b = 11.1420(2) A beta = 90 deg. |
| | c = 21.8188(4) A gamma = 90 deg. |
| Volume | 1336.93(4) [symbol] 3 |
| Z, Calculated density | 4, 1.199 Mg/ [symbol] 3 |
| Absorption coefficient | 0.640 m [symbol] -1 |
| F(000) | 512 |
| Crystal size | 0.20 × 0.07 × 0.04 mm |
| Theta range for data collection | 4.05 to 65.94 deg. |
| Limiting indices | −5<=h<=6, −3<=k<=12, −25<=\|<=25 |
| Reflections collected/unique | 10358/2255 [R(int) = 0.0393] |
| Completeness to theta = 65.94 | 97.1% |
| Absorption correction | Semi-empirical from equivalents |
| Max. and min. transmission | 0.9748 and 0.8827 |
| Refinement method | Full-matrix least-squares on [symbol] 2 |
| Data/restraints/parameters | 2255/0/170 |
| Goodness-of-fit on [symbol] 2 | 1.072 |
| Final R indices [I>2sigma(I)] | R1 = 0.0329, wR2 = 0.0827 |
| R indices (all data) | R1 = 0.0347, wR2 = 0.0840 |
| Absolute structure parameter | 0.3(2) |
| Largest diff. peak and hole | 0.134 and −0.170 e. [symbol] -3 |

TABLE 2

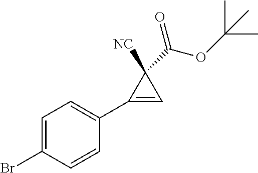 [SMILES: CC(C)(C)OC(=O)[C@]1(C#N)C=C1c1ccc(Br)cc1]

Crystal data and structure refinement for compound 3df

| | |
|---|---|
| Identification code | 3df |
| Empirical formula | C15 H14 Br N O2 |
| Formula weight | 320.18 |
| Temperature | 100(2) K |
| Wavelength | 1.54178 A |
| Crystal system, space group | Orthorhombic, P212121 |
| Unit cell dimensions | a = 5.7660(1) A alpha = 90 deg. |
| | b = 13.9219(2) A beta = 90 deg. |
| | c = 18.1101(2) A gamma = 90 deg. |
| Volume | 1453.76(4) [symbol] 3 |
| Z, Calculated density | 4, 1.463 Mg/ [symbol] 3 |
| Absorption coefficient | 3.835 m [symbol] -1 |
| F(000) | 648 |
| Crystal size | 0.40 × 0.25 × 0.15 mm |
| Theta range for data collection | 4.00 to 67.40 deg. |
| Limiting indices | −6<=h<=5, −16<=k<=16, −21<=\|<=21 |
| Reflections collected/unique | 18467/2596 [R(int) = 0.0448] |
| Completeness to theta = 67.40 | 99.7% |
| Absorption correction | Semi-empirical from equivalents |
| Max. and min. transmission | 0.5970 and 0.3092 |
| Refinement method | Full-matrix least-squares on [symbol] 2 |
| Data/restraints/parameters | 2596/0/175 |
| Goodness-of-fit on [symbol] 2 | 1.094 |
| Final R indices [I>2sigma(I)] | R1 = 0.0208, wR2 = 0.0524 |
| R indices (all data) | R1 = 0.0211, wR2 = 0.0525 |
| Absolute structure parameter | 0.022(15) |
| Largest diff. peak and hole | 0.436 and −0.337 e. [symbol] -3 |

TABLE 3

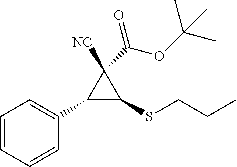 [SMILES: CCCS[C@H]1[C@H](c2ccccc2)[C@]1(C#N)C(=O)OC(C)(C)C]

Crystal data and structure refinement for compound 4a

| | |
|---|---|
| Identification code | 4a |
| Empirical formula | C18 H23 N O2 S |
| Formula weight | 317.43 |
| Temperature | 100(2) K |
| Wavelength | 1.54178 A |
| Crystal system, space group | Orthorhombic, P212121 |
| Unit cell dimensions | a = 5.5837(2) A alpha = 90 deg. |
| | b = 11.9770(4) A beta = 90 deg. |
| | c = 26.7431(9) A gamma = 90 deg. |
| Volume | 1788.47(11) [symbol] 3 |
| Z, Calculated density | 4, 1.179 Mg/ [symbol] 3 |
| Absorption coefficient | 1.652 m [symbol] -1 |
| F(000) | 680 |
| Crystal size | 0.20 × 0.10 × 0.05 mm |
| Theta range for data collection | 3.31 to 66.71 deg. |
| Limiting indices | −5<=h<=6, −14<=k<=14, −30<=\|<=31 |
| Reflections collected/unique | 16860/3121 [R(int) = 0.0488] |
| Completeness to theta = 66.71 | 98.1% |
| Absorption correction | Semi-empirical from equivalents |
| Max. and min. transmission | 0.9220 and 0.7336 |
| Refinement method | Full-matrix least-squares on [symbol] 2 |
| Data/restraints/parameters | 3121/0/304 |
| Goodness-of-fit on [symbol] 2 | 1.049 |
| Final R indices [I>2sigma(I)] | R1 = 0.0347, wR2 = 0.0862 |
| R indices (all data) | R1 = 0.0382, wR2 = 0.0880 |
| Absolute structure parameter | 0.013(18) |
| Largest diff. peak and hole | 0.302 and −0.187 e. [symbol] -3 |

What is claimed is:

1. A cobalt porphyrin complex corresponding to Formula [Co(P2)]

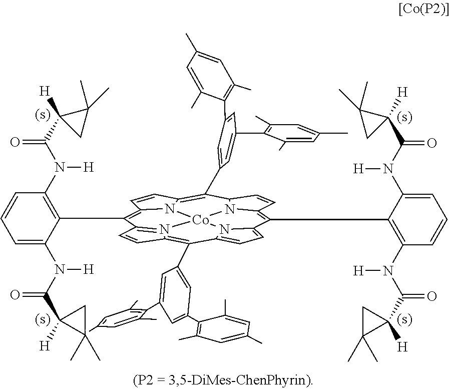

2. A process for the preparation for the preparation of a cyclopropene, the process comprising treating an alkyne with an acceptor/acceptor substituted diazo reagent in the presence of a metal porphyrin complex.

3. The process of claim 2 wherein the metal porphyrin complex is a cobalt(II) complex.

4. The process of claim 2 wherein the metal porphyrin complex is a cobalt(II) complex of a D₂-symmetric chiral porphyrin.

5. The process of claim 2 wherein the metal porphyrin complex corresponds to Formula [Co(P2)].

6. The process of claim 2 wherein the alkyne corresponds to Formula A-1:
R₁—≡—R₂ Formula A-1
wherein R₁ is hydrocarbyl, substituted hydrocarbyl, heterocyclo, or an electron withdrawing group, and R₂ is hydrogen, substituted hydrocarbyl, or heterocyclo.

7. The process of claim 6 wherein R₁ is hydrocarbyl, substituted hydrocarbyl, or heterocyclo.

8. The process of claim 6 wherein R₁ is optionally substituted alkyl or optionally substituted phenyl.

9. The process of any of claim 2 wherein the acceptor/acceptor-substituted diazo reagent corresponds to Formula D-1: 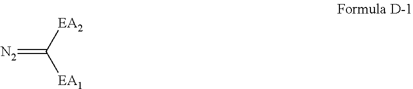
wherein EA₁ and EA₂ are the same or different, and each is an electron-acceptor.

10. The process of claim 9 wherein EA₁ and EA₂ are independently hydroxy, alkoxy, mercapto, halogen, carbonyl, sulfonyl, nitrile, quaternary amine, nitro, trihalomethyl, imine, amidine, oxime, thioketone, thioester, or thioamide.

11. The process of claim 9 wherein EA₁ and EA₂ are independently halogen, carbonyl, nitrile, quaternary amine, nitro, or trihalomethyl.

12. The process of claim 9 wherein EA₁ and EA₂ are independently halogen, carbonyl, nitrile, nitro, or trihalomethyl.

13. The process of claim 9 wherein EA₁ is nitrile and EA₂ is carbonyl.

14. The process of claim 9 wherein the acceptor/acceptor-substituted diazo reagent is an α-cyanodiazoacetamide or an α-cyanodiazoacetate.

15. A cyclopentene corresponding to Formula C-1 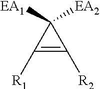
wherein
R₁ is hydrocarbyl, substituted hydrocarbyl, heterocyclo, or an electron withdrawing group,
R₂ is hydrogen, substituted hydrocarbyl, or heterocyclo, and
EA₁ and EA₂ are the same or different, and each is an electron-acceptor.

16. The cyclopentene of claim 15 wherein R₂ is hydrogen.

17. The cyclopentene of claim 15 wherein EA₁ and EA₂ are independently hydroxy, alkoxy, mercapto, halogen, carbonyl, sulfonyl, nitrile, quaternary amine, nitro, trihalomethyl, imine, amidine, oxime, thioketone, thioester, or thioamide.

18. The cyclopentene of claim 15 wherein EA₁ and EA₂ are independently halogen, carbonyl, nitrile, quaternary amine, nitro, or trihalomethyl.

19. The cyclopentene of claim 15 wherein R₁ is hydrocarbyl, substituted hydrocarbyl, or heterocyclo.

20. The cyclopentene of claim 15 wherein R₁ is optionally substituted alkyl or optionally substituted phenyl.
